SEQUENCE LISTING (11 sequences)

SEQ ID NO: 1 - DNA, 21 bp, synthetic construct
  source 1..21: mol_type = other DNA
cgctgagtacttcgaaatgtc

SEQ ID NO: 2 - DNA, 21 bp, synthetic construct
  source 1..21: mol_type = other DNA
caacaagatgaagagcaccaa

SEQ ID NO: 3 - DNA, 21 bp, synthetic construct
  source 1..21: mol_type = other DNA
cgtgatggaccaagccatcaa

SEQ ID NO: 4 - DNA, 21 bp, synthetic construct
  source 1..21: mol_type = other DNA
cccagcatctctgttgctaac

SEQ ID NO: 5 - DNA, 21 bp, synthetic construct
  source 1..21: mol_type = other DNA
gcctctctcccaacaatagtg

SEQ ID NO: 6 - DNA, 21 bp, synthetic construct
  source 1..21: mol_type = other DNA
gtaacctcctccgccacatta

SEQ ID NO: 7 - DNA, 21 bp, synthetic construct
  source 1..21: mol_type = other DNA
cgcgactcctatgcatagaaa

SEQ ID NO: 8 - DNA, 21 bp, synthetic construct
  source 1..21: mol_type = other DNA
gcagactccttctaccagttt

SEQ ID NO: 9 - DNA, 21 bp, synthetic construct
  source 1..21: mol_type = other DNA
caggatagtaaagaagccaaa

SEQ ID NO: 10 - DNA, 21 bp, synthetic construct
  source 1..21: mol_type = other DNA
gtgccagcacaacgtgtgcaa

SEQ ID NO: 11 - DNA, 21 bp, synthetic construct
  source 1..21: mol_type = other DNA
atgtgggatgagacggaattg

(19) United States
(12) Patent Application Publication
(10) Pub. No.: US 2026/0234630 A1
(43) Pub. Date: Aug. 13, 2026

(54) Methods for Treating Multiple Myeloma

(71) Applicant: Celgene Corporation, Princeton, NJ (US)

(72) Inventor: Chih-Chao Hsu, Basking Ridge, NJ (US)

(73) Assignee: Celgene Corporation, Princeton, NJ (US)

(21) Appl. No.: 19/109,255

(22) PCT Filed: Sep. 11, 2023
(86) PCT No.: PCT/US2023/073836

§ 371 Date: Mar. 6, 2025

Related U.S. Application Data
(60) Provisional application No. 63/375,300, filed on Sep. 12, 2022.

(51) Int. Cl. C12N 15/113; A61K 31/136; A61K 31/473; A61K 31/4745; A61K 31/513; A61K 31/704; A61K 45/06; A61P 35/00; C12Q 1/6809; C12Q 1/6886

(52) U.S. Cl. CPC C12N 15/1135; A61K 31/136; A61K 31/473; A61K 31/4745; A61K 31/513; A61K 31/704; A61K 45/06; A61P 35/00; C12Q 1/6809; C12Q 1/6886; C12N 2310/14; C12N 2310/531; C12Q 2600/106

(57) ABSTRACT

Provided herein are methods of treating multiple myeloma, including multiple myeloma that is resistant to at least one therapeutic agent, comprising administering to a subject with multiple myeloma an inhibitor of Ubiquitin-Like with PHD and Ring Finger Domains 1 (UHRF1).

### I. CROSS-REFERENCE TO RELATED APPLICATIONS

This application claims the benefit of priority of U.S. Provisional Application No. 63/375,300, filed Sep. 12, 2022, which is incorporated by reference herein in its entirety for any purpose.

### II. SEQUENCE LISTING

The instant application contains a Sequence Listing, which has been submitted electronically in XML format and is hereby incorporated by reference in its entirety. Said XML copy, created on Sep. 6, 2023, is named “2023-09-06_01277-0017-00PCT_SL” and is 10,716 bytes in size.

### III. FIELD

Provided herein are methods of treating multiple myeloma, comprising administering to a subject with multiple myeloma an inhibitor of Ubiquitin-Like with PHD and Ring Finger Domains 1 (UHRF1). In some embodiments, the multiple myeloma is resistant to at least one therapeutic agent.

### IV. BACKGROUND

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including but not limited to M-protein and other immunoglobulins (antibodies), albumin, and beta-2-microglobulin, except in some patients (estimated at 1% to 5%) whose myeloma cells do not secrete these proteins (termed non-secretory myeloma). M protein, short for monoclonal protein, also known as paraprotein, is a particularly abnormal protein produced by the myeloma plasma cells and can be found in the blood or urine of almost all patients with multiple myeloma, except for patients who have non-secretory myeloma or whose myeloma cells produce immunoglobulin light chains with heavy chain.

Skeletal symptoms, including bone pain, are among the most clinically significant symptoms of multiple myeloma. Malignant plasma cells release osteoclast stimulating factors (including IL-1, IL-6 and TNF) which cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency.

There is a need for improved treatment options for multiple myeloma.

### V. SUMMARY

Provided herein are methods of treating multiple myeloma, comprising administering to a subject with multiple myeloma an inhibitor of Ubiquitin-Like with PHD and Ring Finger Domains 1 (UHRF1). In some embodiments, the multiple myeloma is resistant to at least one therapeutic agent. In some embodiments, the method comprises administering an inhibitor of UHRF1 and at least one additional therapeutic agent.

Also provided are methods of predicting whether a subject with multiple myeloma will benefit from treatment with a UHRF1 inhibitor, comprising determining whether the subject has a UHRF1 level above a reference level.

The following non-limiting embodiments are provided.
Embodiment 1. A method of treating multiple myeloma, comprising administering to a subject with multiple myeloma a therapeutically effective amount of an inhibitor of Ubiquitin-Like with PHD and Ring Finger Domains 1 (UHRF1), wherein the multiple myeloma is resistant to at least one therapeutic agent.
Embodiment 2. The method of embodiment 1, wherein the multiple myeloma is resistant to at least one IMiD®.
Embodiment 3. The method of embodiment 2, wherein the multiple myeloma is resistant to at least one IMiD® selected from lenalidomide, thalidomide, pomalidomide, avadomide, and iberdomide.
Embodiment 4. A method of treating multiple myeloma, comprising administering to a subject with multiple myeloma a therapeutically effective amount of an inhibitor of Ubiquitin-Like with PHD and Ring Finger Domains 1 (UHRF1) and at least one additional therapeutic agent.
Embodiment 5. The method of embodiment 3, wherein the multiple myeloma is resistant to at least one IMiDR.
Embodiment 6. The method of embodiment 5, wherein the multiple myeloma is resistant to at least one IMiD® selected from lenalidomide, thalidomide, pomalidomide, avadomide, and iberdomide.
Embodiment 7. The method of any one of embodiments 4-6, wherein at least one additional therapeutic agent is selected from a chemotherapy agent, a steroid, an immunomodulating agent, a proteasome inhibitor, a histone deacetylase inhibitor, an anti-CD38 antibody, an anti-SLAMF7 antibody, an antibody-drug conjugate, a nuclear export inhibitor, a BCMA-directed T-cell engager, an NK cell engager, and a CAR-T therapy.
Embodiment 8. The method of any one of embodiments 4-7, wherein at least one additional therapeutic agent is selected from lenalidomide, thalidomide, pomalidomide, avadomide, iberdomide, bortezomib, carfilzomib, ixazomib, panobinostat, melphalan, vincristine, cyclophosphamide, etoposide, doxorubicin, and bendamustine, dexamethasone, prednisone, daratumumab, isatuximab, elotuzumab, belantamab mafodotin-blmf, Selinexor, pamidronate, zoledronic acid, and denosumab.
Embodiment 9. The method of any one of embodiments 4-8, wherein the at least one additional therapeutic agent is selected from:
   a) lenalidomide;
   b) iberdomide;
   c) (S)-4-(4-(4-(((2-(2,6-dioxopiperidin-3-yl)-1-oxoisoindolin-4-yl)oxy)methyl)benzyl) piperazin-1-yl)-3-fluorobenzonitrile;
   d) (i) lenalidomide, pomalidomide, or thalidomide; and (ii) dexamethasone;
   e) (i) carfilzomib, ixazomib, or bortezomib; (ii) lenalidomide; and (iii) dexamethasone;
   f) (i) bortezomib or carfilzomib; (ii) cyclophosphamide; and (iii) dexamethasone;
   g) (i) elotuzumab or daratumumab; (ii) lenalidomide; and (iii) dexamethasone;
   h) bortezomib, liposomal doxorubicin, and dexamethasone;
   i) panobinostat, bortezomib, and dexamethasone;
   j) elotuzumab, bortezomib, and dexamethasone;
   k) melphalan and prednisone, with or without thalidomide or bortezomib;
   l) vincristine, doxorubicin, and dexamethasone;
   m) dexamethasone, cyclophosphamide, etoposide, and cisplatin; and
   n) dexamethasone, thalidomide, cisplatin, doxorubicin, cyclophosphamide, and etoposide, with or without bortezomib.
Embodiment 10. The method of any one of the preceding embodiments, wherein the inhibitor of UHRF1 is not an IMiDR.
Embodiment 11. The method of any one of the preceding embodiments, wherein the inhibitor of UHRF1 is a direct inhibitor of UHRF1.
Embodiment 12. The method of embodiment 11, wherein the direct inhibitor of UHRF1 is a direct inhibitor of a UHRF1 protein.
Embodiment 13. The method of embodiment 11 or embodiment 12, wherein the inhibitor of UHRF1 is a small molecule, aptamer, or antibody that binds to the UHRF1 protein.
Embodiment 14. The method of any one of embodiments 11-13, wherein the inhibitor of UHRF1 is idarubicin, mitoxantrone, berberine, daunorubicin, doxorubicin, mitoxantrone, pixantrone, 2,4-lutidine, or NSC232003.
Embodiment 15. The method of embodiment 11, wherein the inhibitor of UHRF1 is a direct inhibitor of a UHRF1 transcript.
Embodiment 16. The method of embodiment 15, wherein the inhibitor of UHRF1 comprises an oligonucleotide that hybridizes to the UHRF1 transcript.
Embodiment 17. The method of embodiment 15 or embodiment 16, wherein the inhibitor of UHRF1 comprises an siRNA, shRNA, or antisense oligonucleotide.
Embodiment 18. The method of any one of the preceding embodiments, wherein the multiple myeloma cells express a UHRF1 transcript or protein.
Embodiment 19. The method of embodiment 18, wherein the multiple myeloma expresses elevated levels of the UHRF1 transcript or protein relative to a reference level of UHRF1 transcript or protein.
Embodiment 20. The method of embodiment 18 or embodiment 19, wherein the UHRF1 transcript or protein expression is determined by a method comprising in situ hybridization, PCR, RNA sequencing, fluorescence in situ hybridization (FISH), mixed ligation probe assays, RT-qPCR, western blot, ELISA, or mass spectrometry.
Embodiment 21. A method of selecting a subject with multiple myeloma for treatment with a UHRF1 inhibitor, comprising determining the level of UHRF1 in a sample obtained from the subject, wherein if the level of UHRF1 in the sample is above a reference level, the subject is selected for treatment with a UHRF1 inhibitor.
Embodiment 22. A method of predicting whether a subject with multiple myeloma will benefit from treatment with a UHRF1 inhibitor, comprising determining whether the subject has a level of UHRF1 above a reference level.
Embodiment 23. The method of embodiment 22, wherein the level of UHRF1 is determined in a sample from the subject.
Embodiment 24. The method of any one of embodiments 21-23, wherein the level of UHRF1 is the level of a UHRF1 transcript or protein.
Embodiment 25. The method of embodiment 24, wherein the UHRF1 transcript or protein level is determined by a method comprising in situ hybridization, PCR, RNA sequencing, fluorescence in situ hybridization (FISH), mixed ligation probe assays, RT-qPCR, western blot, ELISA, or mass spectrometry.
Embodiment 26. The method of any one of embodiments 21-25, wherein the method further comprises administering to the subject a therapeutically effective amount of an inhibitor of UHRF1.
Embodiment 27. The method of embodiment 26, wherein the multiple myeloma is resistant to at least one IMiD®.
Embodiment 28. The method of embodiment 27, wherein the multiple myeloma is resistant to at least one IMiD® selected from lenalidomide, thalidomide, pomalidomide, avadomide, and iberdomide.
Embodiment 29. A method of treating multiple myeloma, comprising administering to a subject with multiple myeloma a therapeutically effective amount of an inhibitor of Ubiquitin-Like with PHD and Ring Finger Domains 1 (UHRF1) and at least one additional therapeutic agent.
Embodiment 30. The method of embodiment 29, wherein the multiple myeloma is resistant to at least one IMiDR.
Embodiment 31. The method of embodiment 30, wherein the multiple myeloma is resistant to at least one IMiD® selected from lenalidomide, thalidomide, pomalidomide, avadomide, and iberdomide.
Embodiment 32. The method of any one of embodiments 29-31, wherein at least one additional therapeutic agent is selected from a chemotherapy agent, a steroid, an immunomodulating agent, a proteasome inhibitor, a histone deacetylase inhibitor, an anti-CD38 antibody, an anti-SLAMF7 antibody, an antibody-drug conjugate, a nuclear export inhibitor, a BCMA-directed T-cell engager, an NK cell engager, and a CAR-T therapy.
Embodiment 33. The method of any one of embodiments 29-32, wherein at least one additional therapeutic agent is selected from lenalidomide, thalidomide, pomalidomide, iberdomide, bortezomib, carfilzomib, ixazomib, panobinostat, melphalan, vincristine, cyclophosphamide, etoposide, doxorubicin, and bendamustine, dexamethasone, prednisone, daratumumab, isatuximab, elotuzumab, belantamab mafodotin-blmf, Selinexor, pamidronate, zoledronic acid, and denosumab.
Embodiment 34. The method of any one of embodiments 29-33, wherein the at least one additional therapeutic agent is selected from:
   a) lenalidomide;
   b) iberdomide;
   c) (S)-4-(4-(4-(((2-(2,6-dioxopiperidin-3-yl)-1-oxoisoindolin-4-yl)oxy)methyl)benzyl) piperazin-1-yl)-3-fluorobenzonitrile;
   d) (i) lenalidomide, pomalidomide, or thalidomide; and (ii) dexamethasone;
   e) (i) carfilzomib, ixazomib, or bortezomib; (ii) lenalidomide; and (iii) dexamethasone;
   f) (i) bortezomib or carfilzomib; (ii) cyclophosphamide; and (iii) dexamethasone;
   g) (i) elotuzumab or daratumumab; (ii) lenalidomide; and (iii) dexamethasone;
   h) bortezomib, liposomal doxorubicin, and dexamethasone;
   i) panobinostat, bortezomib, and dexamethasone;
   j) elotuzumab, bortezomib, and dexamethasone;
   k) melphalan and prednisone, with or without thalidomide or bortezomib;
   l) vincristine, doxorubicin, and dexamethasone;
   m) dexamethasone, cyclophosphamide, etoposide, and cisplatin; and
   n) dexamethasone, thalidomide, cisplatin, doxorubicin, cyclophosphamide, and etoposide, with or without bortezomib.
Embodiment 35. The method of any one of embodiments 26-34, wherein the inhibitor of UHRF1 is not an IMiD®.
Embodiment 36. The method of any one of embodiments 26-35, wherein the inhibitor of UHRF1 is a direct inhibitor of UHRF1.
Embodiment 37. The method of embodiment 36, wherein the direct inhibitor of UHRF1 is a direct inhibitor of a UHRF1 protein.
Embodiment 38. The method of embodiment 36 or embodiment 37, wherein the inhibitor of UHRF1 is a small molecule, aptamer, or antibody that binds to the UHRF1 protein.
Embodiment 39. The method of any one of embodiments 36-38, wherein the inhibitor of UHRF1 is idarubicin, mitoxantrone, berberine, daunorubicin, doxorubicin, mitoxantrone, pixantrone, 2,4-lutidine, or NSC232003.
Embodiment 40. The method of embodiment 36, wherein the inhibitor of UHRF1 is a direct inhibitor of a UHRF1 transcript.
Embodiment 41. The method of embodiment 40, wherein the inhibitor of UHRF1 comprises an oligonucleotide that hybridizes to the UHRF1 transcript.
Embodiment 42. The method of embodiment 40 or embodiment 41, wherein the inhibitor of UHRF1 comprises an siRNA, shRNA, or antisense oligonucleotide.

a. The method of any one of embodiments 21-42, wherein the sample is a blood sample, plasma sample, or multiple myeloma sample.
Embodiment 43. The method of embodiment 43, wherein the sample is a multiple myeloma sample.

### VI. BRIEF DESCRIPTION OF THE DRAWINGS

FIG. 1A-1F. Down regulation of UHRF1 transcription by IMiDs correlates with drug sensitivity of multiple myeloma. (1A) Volcano plot of protein expression changes determined by mass spectrometry in H929 cells at 24 hr of treatment with CC-220 (0.1 μM) or DMSO control. X axis, Log2fold change (FC); Y axis, significance,-log 10 [p-value]. Representative top downregulated and upregulated proteins are highlighted. (1B) Volcano plot of gene expression changes determined by Affymetrix microarray in H929 cells treated with POM (1 μM) or DMSO for 24 hr. X axis, Log2fold change (FC); Y axis, significance,-log 10 [p-value], n=2 technical replicates). Representative top downregulated and upregulated genes are highlighted. (1C) Western blot analysis protein expression of UHRF1, Ikaros, Aiolos and ZFP91 in H929 cells treated with 1 μM POM for indicated time. (1D) qRT-PCR analysis of UHRF1 gene expression levels in cells as in (1C). Error bars represent the SEM from duplicate measurements. (1E) Western blot analysis protein expression of UHRF1 and indicated proteins in MM cells treated with DMSO (−) or 1 μM POM (+) for 3 days. GAPDH serves as a loading control. (1F) Thymidine incorporation of various MM cell lines treated with indicated dose of POM for 3 days. Error bars represent the SEM from technical replicates.

FIG. 2A-2E. UHRF1 expression is positively regulated by Ikaros and Aiolos in IMiDs sensitive MM cells. (2A) Western blot analysis protein expression of UHRF1, Ikaros, Aiolos, ZFP91 and Cereblon expression in control [shLuciferase (shLuc)] and cereblon knockdown (shCRBN) H929 cells treated with DMSO and 1 μM POM for 3 days. GAPDH, loading control. Arrow head, UHRF1. (2B) qRT-PCR analysis of UHRF1 gene expression levels in cells as in (2A). Data was normalized to ACTB. Error bars represent the SEM of technical duplicates. *** P<0.001 (Two-tailed unpaired Student's t-test). (2C) Western blot analysis protein expression of UHRF1, Ikaros and Aiolos in stably transduced H929 cells after Dox induction (0, 10, 50 or 100 ng/ml) for 3 days of non-target shRNA (shNT) or shRNAs targeting Ikaros (shIKZF1) or Aiolos (shIKZF3). (2D) qRT-PCR analysis of UHRF1 gene expression levels in cells as in (2C). Shown are cells induced with Dox (50 ng/ml) for 3 days. Data was normalized to GAPDH. Error bars represent the SEM of technical duplicates. *P<0.05, ** P<0.01, *** P<0.001, **** P<0.0001 (Two-tailed unpaired Student's t-test). (2E) ChIP-qPCR analysis of the occupancy Aiolos (upper panel) or H3K27ac (lower panel) occupancy at transcription star site (TSS), gene body (+7 kb) and gene end (GE) of UHRF1 gene in H929, MM1.S or RPMI 8226 cells treated with DMSO or 1 μM POM for 24 hr. ChIP with control rabbit IgG (IgG) serves as a negative control. Error bars represent the SEM of technical triplicates. * P<0.05, ** P<0.01, **** P<0.0001 (Two-tailed unpaired Student's t-test).

FIG. 3A-3E. UHRF1 is essential for cell proliferation and survival of MM cells. (3A) Western blot analysis of UHRF1 knockdown efficiency. H929 cells stably transduced with shNT or indicated shRNAs targeting UHRF1 were induced with 20 ng/ml of Dox for 6 days. (3B) Cell proliferation assays of control (shNT) or UHRF1 knockdown (shUHRF1) H929 cells. Stably transduced H929 cells were induced with Dox (20 ng/ml) to express indicated shRNA for 3 days. Cells were then seeded at 2×10⁵ cells/ml and cell number were counted for 5 consecutive days. Error bars represent the SEM from duplicate measurements. (3C) Apoptosis assay of control (shNT) or UHRF1 knockdown (shUHRF1) H929 cells. Stably transduced H929 cells were induced to express indicated shRNA for 5 and 7 days, followed by analyzing apoptosis with Annexin V and TO-PRO-3 staining. Number in each corner represent the percentage of cells in that quadrant. (3D) Cell cycle assay of control (shNT) or UHRF1 knockdown (shUHRF1) H929 cells. Cell cycle profiles were analyzed on day 7 of knockdown. (3E) Stacked bar graphs showing the percentage of cells at various stages of cell cycle quantified from (3D).

FIG. 4A-4C. UHRF1 depletion induces interferon response genes. (4A) Venn diagrams showing overlaps of upregulated genes (log 2FC ≥0.5 and FDR≤0.05, left panel) or downregulated genes (log 2FC≤−0.5 and FDR≤0.05, right panel) in two UHRF1 KD H929 cells (shUHRF1-4 and shUHRF1-15) compared with the control cells (shNT). (4B) GO analysis of overlapping upregulated genes in shUHRF1-4 and shUHRF1-15 treated H929 cells. The top 14 enriched process are listed. (4C) GSEA plots evaluating the changes in Interferon alpha response gene signatures in H929 cells treated with shUHRF1-15 (left panel) and shUHRF1-4 (right panel). NES, normalized enrichment score; FDR, false discovery rate.

FIG. 5A-5D. High UHRF1 expression is associated with poor prognosis in MM patients. (5A) Overall survival of ndMM patients with high or low UHRF1 expression. (5B) Disease free survival of ndMM patients with high or low UHRF1 expression. (5C) Overall survival of rrMM patients with high or low UHRF1 expression. (5D) Disease free survival of rrMM patients with high or low UHRF1 expression.

FIG. 6A-6D. UHRF1 gene expression is downregulated by pomalidomide and CC-220 in drug sensitive MM. (6A) Immunocytochemistry assay of UHRF1 in H929 cells treated with DMSO or 1 μM POM for 48 hrs. (6B) qRT-PCR analysis of UHRF1 gene expression levels in indicated MM cells treated with indicated doses of POM for 3 days. Data was normalized to ACTB. Error bars represent the SEM from duplicate measurements. * P<0.05, ** P<0.01, *** P<0.001, **** P<0.0001 (Two-tailed Student's t-test). (6C) Western blot analysis protein expression of UHRF1, Ikaros, Aiolos, ZFP91 and Celebron in H929 cells treated with 1 μM POM or 0.1 μM CC-220 for 3 days. DMSO, vehicle control. (6D) Western blot analysis protein expression of UHRF1, Ikaros, Aiolos, ZFP91 and Celebron in H929 or RPMI 8226 cells treated with 1 μM POM, 0.1 μM CC-220 or 0.01 μM CC-480 for 3 days. DMSO, vehicle control.

FIG. 7A-7B. UHRF1 expression is positively regulated by Ikaros, Aiolos and ZFP91 in MM1.S cells. (7A) Western blot analysis protein expression of UHRF1, Ikaro and Aiolos in stably transduced MM1.S cells after Dox induction (0, 20, or 50 ng/ml) for 3 days of non-target shRNA (shNT) or shRNAs targeting Ikaros (shIKZF1) or Aiolos (shIKZF3). (7B) Western blot analysis histone H3 and H3K27ac levels from H929 and MM1.S cells treated with DMSO or 1 μM POM for 24 hr.

FIG. 8A-8H. UHRF1 is essential for MM1.S and RPMI 8226 cells. (8A) Western blot analysis of UHRF1 knockdown efficiency. MM1.S cells stably transduced with shNT or indicated shRNAs targeting UHRF1 were induced with 10 ng/ml of Dox for 4 days. (8B) Cell proliferation assays of control (shNT) or UHRF1 knockdown (shUHRF1) H929 cells. Stably transduced MM1.S cells were induced with Dox (10 ng/ml) to express indicated shRNA for 3 days. Cells were then seeded at 4×10⁵ cells/ml and cell number were counted for up to 5 days. Error bars represent the SEM from duplicate measurements. (8C) Apoptosis assay of control (shNT) or UHRF1 knockdown (shUHRF1) MM1.S cells. Stably transduced MM1.S cells were induced to express indicated shRNA for 4 and 5 days, followed by analyzing apoptosis with Annexin V and TO-PRO-3 staining. Number in each corner represent the percentage of cells in that quadrant. (8D) Cell cycle assay of control (shNT) or UHRF1 knockdown (shUHRF1) MM1.S cells. Cell cycle profiles were analyzed on day 4 and 7 of knockdown. (8E) Stacked bar graphs showing the percentage of MM1.S cells at various stages of cell cycle quantified from (8D). (8F) Western blot analysis of UHRF1 knockdown efficiency. RPMI 8226 cells stably transduced with shNT or indicated shRNAs targeting UHRF1 were induced with 20 ng/ml of Dox for 5 days. (8G) Cell proliferation assays of control (shNT) or UHRF1 knockdown (shUHRF1) H929 cells. Stably transduced RPMI 8226 cells were induced with Dox (10 ng/ml) to express indicated shRNA for 3 days. Cells were then seeded at 4×10⁵ cells/ml and cell number were counted for up to 5 days. Error bars represent the SEM from duplicate measurements. (8H) Apoptosis assay of control (shNT) or UHRF1 knockdown (shUHRF1) RPMI 8226 cells. Stably transduced MM1.S cells were induced to express indicated shRNA for 5 days, followed by analyzing apoptosis with Annexin V and TO-PRO-3 staining. Number in each corner represent the percentage of cells in that quadrant.

### VII. DETAILED DESCRIPTION OF THE INVENTION

### A. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of.” Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C.” An exception to this definition will occur only when a combination of elements, functions, steps or acts are in some way inherently mutually exclusive.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, a “Direct Inhibitor” is an inhibitor that effects inhibition of its target by a mechanism that comprises binding to the target. An inhibitor that effects inhibition of a target downstream of the molecule to which it binds is not a direct inhibitor of that target.

Unless otherwise indicated, “UHRF1” is any UHRF1 protein (full length, truncated, isoform, or pre-processed or processed form) that is expressed in a non-genetically modified cell, or any molecule that encodes such a UHRF1 protein, such as a UHRF1 gene, UHRF1 pre-mRNA, or UHRF1 mRNA.

“IMiD” or “immunomodulatory drug that binds to cereblon” is an immunomodulatory drug that is a member of the group of therapeutics that binds cereblon and triggers degradation of one or more cereblon substrates. Nonlimiting exemplary IMiDs include lenalidomide, thalidomide, pomalidomide, avadomide, and iberdomide.

“Inhibitor” reduces activity and/or expression of a target by a detectable amount.

“Reference level” is a level or range of levels associated with a reference condition. For example, the reference level of a protein in a sample from a subject may be the level or range of levels within which the protein in a sample from a healthy subject is expected to be found.

As used herein and unless otherwise indicated, the term “express” and “expression” refer to gene expression, include expression of nucleic acids (e.g., , mRNA) and expression of polypeptides. The term “level” refers to a level of an mRNA and/or a level of a polypeptide. Thus, “UHRF1 expression” can be determined by evaluating expression of UHRF1 mRNA and/or expression of UHRF1 protein, and the “level of UHRF1” may be the level of the UHRF1 mRNA and/or the level of the UHRF1 protein.

As used herein and unless otherwise indicated, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein and unless otherwise indicated, the term “preventing” means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein and unless otherwise indicated, the term “effective amount” or “therapeutically effective amount” in connection with a compound means an amount capable of treating, preventing, or managing a disorder, disease or condition, or symptoms thereof.

As used herein and unless otherwise indicated, the term “subject” or “patient” includes an animal, and in some embodiments, a mammal. In some embodiments, a subject or patient is a human.

As used herein and unless otherwise indicated, the term “sample” refers to a sample obtained from a subject. The sample may be from any biological tissue or fluid. In some embodiments, a sample is derived from a human, e.g., a subject or a patient, e.g., a cancer patient, e.g., a multiple myeloma patient. A sample may include tissues, sections of tissues, cells, fluids, or extracts thereof, and can be isolated by any means, e.g., from blood, serum, biopsy, lymph node biopsy, bone marrow biopsy, needle biopsy, aspiration, etc.).

As used herein and unless otherwise indicated, the term “relapsed” refers to a disorder, disease, or condition that responded to treatment (e.g., achieved a partial or complete response) then had progression. The treatment can include one or more lines of therapy. In some embodiments, the disorder, disease or condition has been previously treated with one or more lines of therapy. In another embodiment, the disorder, disease or condition has been previously treated with one, two, three or four lines of therapy. In some embodiments, the disorder, disease or condition is a hematological malignancy.

As used herein and unless otherwise indicated, the term “refractory” refers to a disorder, disease, or condition that has not responded to prior treatment. In some embodiments, the disorder, disease, or condition has been previously treated one, two, three or four lines of therapy. In some embodiments, the disorder, disease, or condition has been previously treated with two or more lines of treatment, and did not respond to the most recent treatment. In some embodiments, the disorder, disease or condition is a hematological malignancy, and in particular, multiple myeloma.

In the context of a cancer, for example, a hematological malignancy, inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from treatment onset until death from any cause. TTP as used herein means the time from treatment onset until tumor progression; TTP does not include deaths. In some embodiments, PFS means the time from treatment onset until tumor progression or death. In some embodiments, PFS means the time from the first dose of compound to the first occurrence of disease progression or death from any cause. In some embodiments, PFS rates are computed using the Kaplan-Meier estimates. Event-free survival (EFS) means the time from treatment onset until any treatment failure, including disease progression, treatment discontinuation for any reason, or death. In some embodiments, overall response rate (ORR) means the percentage of patients who achieve a response. In some embodiments, ORR means the sum of the percentage of patients who achieve complete and partial responses. In some embodiments, ORR means the percentage of patients whose best response≥partial response (PR). In some embodiments, duration of response (DoR) is the time from achieving a response until relapse or disease progression. In some embodiments, DoR is the time from achieving a response≥partial response (PR) until relapse or disease progression. In some embodiments, DoR is the time from the first documentation of a response until to the first documentation of progressive disease or death. In some embodiments, DoR is the time from the first documentation of a response≥partial response (PR) until to the first documentation of progressive disease or death. In some embodiments, time to response (TTR) means the time from the first dose of compound to the first documentation of a response. In some embodiments, TTR means the time from the first dose of compound to the first documentation of a response ≥ partial response (PR). In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

As used herein “multiple myeloma” refers to hematological conditions characterized by malignant plasma cells and includes the following disorders: monoclonal gammopathy of undetermined significance (MGUS); low risk, intermediate risk, and high risk multiple myeloma; newly diagnosed multiple myeloma (including low risk, intermediate risk, and high risk newly diagnosed multiple myeloma); transplant eligible and transplant ineligible multiple myeloma; smoldering (indolent) multiple myeloma (including low risk, intermediate risk, and high risk smouldering multiple myeloma); active multiple myeloma; solitary plasmacytoma; extramedullary plasmacytoma; plasma cell leukemia; central nervous system multiple myeloma; light chain myeloma; non-secretory myeloma; Immunoglobulin D myeloma; and Immunoglobulin E myeloma; and multiple myeloma characterized by genetic abnormalities, such as Cyclin D translocations (for example, t (11;14) (q13;q32); t (6;14) (p21;32); t (12;14) (p13;q32); or t (6;20);); MMSET translocations (for example, t (4;14) (p16;q32)); MAF translocations (for example, t (14;16) (q32;q32); t (20;22); t (16; 22) (q11;q13); or t (14;20) (q32;q11)); or other chromosome factors (for example, deletion of 17p13, or chromosome 13; del (17/17p), nonhyperdiploidy, and gain (1q)). In some embodiments, the multiple myeloma is characterized by a chromosomal translocation t (4;14). In some embodiments, the multiple myeloma is characterized according to the multiple myeloma International Staging System (ISS). In some embodiments, the multiple myeloma is Stage I multiple myeloma as characterized by ISS (e.g., serum β2 microglobulin <3.5 mg/L and serum albumin ≥3.5 g/dL). In some embodiments, the multiple myeloma is Stage III multiple myeloma as characterized by ISS (e.g., serum β2 microglobulin >5.4 mg/L). In some embodiments, the multiple myeloma is Stage II multiple myeloma as characterized by ISS (e.g., not Stage I or III).

In certain embodiments, the treatment of multiple myeloma may be assessed by the International Uniform Response Criteria for Multiple Myeloma (IURC) (see Durie BGM, Harousseau J-L, Miguel J S, et al. International uniform response criteria for multiple myeloma. Leukemia, 2006; (10) 10:1-7), using the response and endpoint definitions shown below:

| Response | |
|---|---|
| Subcategory | Response Criteria^{a} |
| sCR | CR as defined below plus |
| | Normal FLC ratio and |
| | Absence of clonal cells in bone marrow^{b} by |
| | immunohistochemistry or immunofluorescence^{c} |
| CR | Negative immunofixation on the serum and urine |
| | and Disappearance of any soft tissue |
| | plasmacytomas and <5% plasma cells |
| | in bone marrow^{b} |
| VGPR | Serum and urine M-protein detectable |
| | by immunofixation but not on electrophoresis |
| | or 90% or greater reduction in serum M-protein |
| | plus urine M-protein level <100 mg per 24 h |
| PR | ≥50% reduction of serum M-protein and reduction |
| | in 24-h urinary M-protein by >90% or to <200 mg |
| | per 24 h If the serum and urine M-protein are |
| | unmeasurable,^{d} a ≥50% decrease in the |
| | difference between involved and uninvolved |
| | FLC levels is required in place |
| | of the M-protein criteria |
| | If serum and urine M-protein are unmeasurable, |
| | and serum free light assay is also |
| | unmeasurable, ≥50% reduction in plasma cells is |
| | required in place of M-protein, provided baseline |
| | bone marrow plasma cell percentage was ≥30% |
| | In addition to the above listed criteria, if present |
| | at baseline, a ≥50% reduction in the size of soft |
| | tissue plasmacytomas is also required |
| SD (not | Not meeting criteria for CR, VGPR, |
| recommended for | PR or progressive disease |
| use as an indicator | |
| of response; stability | |
| of disease is best | |
| described by | |
| providing the time | |
| to progression | |
| estimates) | |

Abbreviations:
CR, complete response;
FLC, free light chain;
PR, partial response;
SD, stable disease;
sCR, stringent complete response;
VGPR, very good partial response.
^{a} All response categories require two consecutive assessments made at any time before the institution of any new therapy; all categories also require no known evidence of progressive or new bone lesions if radiographic studies were performed. Radiographic studies are not required to satisfy these response requirements.
^{b}Confirmation with repeat bone marrow biopsy not needed.
^{c}Presence/absence of clonal cells is based upon the κ/λ ratio. An abnormal κ/λ ratio by immunohistochemistry and/or immunofluorescence requires a minimum of 100 plasma cells for analysis. An abnormal ratio reflecting presence of an abnormal clone is κ/λ of >4:1 or <1:2.
^{d}Measurable disease defined by at least one of the following measurements: Bone marrow plasma cells ≥30%; Serum M-protein ≥1 g/dl (≥10 gm/1)[10 g/1]; Urine M-protein ≥200 mg/24 h; Serum FLC assay: Involved FLC level ≥10 mg/dl (≥100 mg/l); provided serum FLC ratio is abnormal.

As used herein, ECOG status refers to Eastern Cooperative Oncology Group (ECOG) Performance Status (Oken M, et al Toxicity and response criteria of the Eastern Cooperative Oncology Group. Am J Clin Oncol 1982;5 (6): 649-655), as shown below:

| Score | Description |
|---|---|
| 0 | Fully active, able to carry on all pre-disease |
| | performance without restriction |
| 1 | Restricted in physically strenuous activity but |
| | ambulatory and able to carry out work of a light or |
| | sedentary nature, eg, light housework, office work. |
| 2 | Ambulatory and capable of all self-care |
| | but unable to carry out any work |
| | activities. Up and about more than 50% of waking hours. |
| 3 | Capable of only limited self-care, confined to bed or |
| | chair more than 50% of waking hours. |
| 4 | Completely disabled. Cannot carry on any self-care. |
| | Totally confined to bed or chair |
| 5 | Dead |

In certain embodiments, stable disease or lack thereof can be determined by methods known in the art such as evaluation of patient symptoms, physical examination, visualization of the tumor that has been imaged, for example using FDG-PET (fluorodeoxyglucose positron emission tomography), PET/CT (positron emission tomography/computed tomography) scan, MRI (magnetic resonance imaging) of the brain and spine, CSF (cerebrospinal fluid), ophthalmologic exams, vitreal fluid sampling, retinal photograph, bone marrow evaluation and other commonly accepted evaluation modalities.

As used herein and unless otherwise indicated, the terms “co-administration” and “in combination with” include the administration of one or more therapeutic agents (for example, a compound provided herein and another anti-cancer agent or supportive care agent) simultaneously, concurrently, or sequentially with no specific time limits. In some embodiments, the agents are present in the cell or in the patient's body at the same time or exert their biological or therapeutic effect at the same time. In some embodiments, the therapeutic agents are in the same composition or unit dosage form. In other embodiments, the therapeutic agents are in separate compositions or unit dosage forms.

The term “supportive care agent” refers to any substance that treats, prevents or manages an adverse effect from treatment with another therapeutic agent.

As used herein, “induction therapy” refers to the first treatment given for a disease, or the first treatment given with the intent of inducing complete remission in a disease, such as cancer. When used by itself, induction therapy is the one accepted as the best available treatment. If residual cancer is detected, patients are treated with another therapy, termed reinduction. If the patient is in complete remission after induction therapy, then additional consolidation and/or maintenance therapy is given to prolong remission or to potentially cure the patient.

As used herein, “consolidation therapy” refers to the treatment given for a disease after remission is first achieved. For example, consolidation therapy for cancer is the treatment given after the cancer has disappeared after initial therapy. Consolidation therapy may include radiation therapy, stem cell transplant, or treatment with cancer drug therapy. Consolidation therapy is also referred to as intensification therapy and post-remission therapy.

As used herein, “maintenance therapy” refers to the treatment given for a disease after remission or best response is achieved, in order to prevent or delay relapse. Maintenance therapy can include chemotherapy, hormone therapy or targeted therapy.

“Remission” as used herein, is a decrease in or disappearance of signs and symptoms of a cancer, for example, multiple myeloma. In partial remission, some, but not all, signs and symptoms of the cancer have disappeared. In complete remission, all signs and symptoms of the cancer have disappeared, although the cancer still may be in the body.

As used herein “transplant” refers to high-dose therapy with stem cell rescue. Hematopoietic (blood) or bone marrow stem cells are used not as treatment but to rescue the patient after the high-dose therapy, for example high dose chemotherapy and/or radiation. Transplant includes “autologous” stem cell transplant (ASCT), which refers to use of the patients' own stem cells being harvested and used as the replacement cells. In some embodiments, transplant also includes tandem transplant or multiple transplants.

The term “biological therapy” refers to administration of biological therapeutics such as cord blood, stem cells, growth factors and the like.

### B. Selection of Subjects for Treatment

In some embodiments, provided herein are methods of treating multiple myeloma, comprising administering to a subject with multiple myeloma an inhibitor of UHRF1.

In some embodiments, provided herein are methods of selecting a subject with multiple myeloma for treatment with an inhibitor of UHRF1, comprising determining whether the subject has a level of UHRF1 above a reference level. In some embodiments, if the subject has an elevated level of UHRRF1, the subject is selected for treatment with an inhibitor of UHRF1.

In some embodiments, provided herein are methods of predicting whether a subject with multiple myeloma will benefit from treatment with an inhibitor of UHRF1, comprising determining whether the subject has a level of UHRF1 above a reference level. In some embodiments, if the subject has a level of UHRF1 above a reference level, the subject is predicted to benefit from treatment with an inhibitor of UHRF1.

The subject may be identified as having multiple myeloma by any available method. In some embodiments, the subject has previously been determined to have multiple myeloma.

In some embodiments, the level of UHRF1 is the level of UHRF1 mRNA. In some embodiments, the level of UHRF1 is the level of UHRF1 protein. In some embodiments, UHRF1 mRNA or protein expression is determined and compared to a reference (e.g., a reference sample, or a reference value, or any other comparison to which is indicative of whether, or to what extent, and/or in what form, the cancer expresses the UHRF1 mRNA or protein). In some embodiments, UHRF1 expression in a multiple myeloma is determined, relative to UHRF1 expression in a non-cancerous cell.

In some embodiments, analyzing and quantifying UHRF1 protein expression in patient samples, cells, and/or cell lines is determined by immunohistochemical and/or immunofluorescence techniques.

In some embodiments, UHRF1 expression is determine by a method comprising polymerase chain reaction (PCR), reverse-transcription-PCR (RT-PCR, including real-time RT-PCR, qRT-PCR), in situ hybridization (ISH), fluorescence in situ hybridization (FISH), transcript in situ hybridization, multiplex ligation-dependent probe assays (MLPA), mass spectrometry (MS), and/or matrix assisted laser desorption ionization-time of flight MS (MALDI-TOF MS).

The term “polymerase chain reaction,” or “PCR,” as used herein refers to a procedure wherein small amounts of a nucleic acid, RNA and/or DNA, are amplified. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc.

Other PCR-based methods can also be used. Examples of PCR methods can be found in the literature. Examples of PCR assays can be found, for example, in U.S. Pat. No. 6,927,024. Nonlimiting examples of RT-PCR methods can be found in U.S. Pat. No. 7,122,799. A nonlimiting method of fluorescent in situ PCR is described in U.S. Pat. No. 7,186,507.

In some embodiments, Real-Time Reverse Transcription-PCR (qRT-PCR) can be used for both the detection and quantification of RNA targets (Bustin, et al., 2005, Clin. Sci., 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663.

In contrast to regular reverse transcriptase-PCR and analysis by agarose gels, real-time PCR gives quantitative results. An additional advantage of real-time PCR is the relative ease and convenience of use. Instruments for real-time PCR, such as the Applied Biosystems 7500, are available commercially, as are the reagents, such as TaqMan Sequence Detection chemistry. For example, TaqMan® Gene Expression Assays can be used, following the manufacturer's instructions. These kits are pre-formulated gene expression assays for rapid, reliable detection and quantification of human, mouse and rat mRNA transcripts. An exemplary PCR program, for example, is 50° C. for 2 minutes, 95° C. for 10 minutes, 40 cycles of 95° C. for 15 seconds, then 60° C. for 1 minute.

The term “in situ hybridization” or “ISH” refers to techniques that can be used to determine mRNA levels (reviewed by A. K. Raap (1998) Mutat. Res. 400:287-298). In situ hybridization techniques allow the visual detection of mRNA in a cell by incubating the cell with a labeled (e.g., fluorescently labeled or digoxigenin labeled) oligonucleotide probe that hybridizes to the mRNA of interest, and then examining the cell by microscopy.

The term “mass spectrometry” or “mass spec” or “MS” as used herein, refers to an analytical technique for measuring the mass-to-charge ratio of ions. This is achieved by ionizing the sample and separating ions of differing masses and recording their relative abundance by measuring intensities of ion flux. A typical mass spectrometer comprises three parts: an ion source, a mass analyzer, and a detector system. The ion source is the part of the mass spectrometer that ionizes the substance under analysis (the analyte). The ions are then transported by magnetic or electric fields to the mass analyzer that separates the ions according to their mass-to-charge ratio (m/z). Many mass spectrometers use two or more mass analyzers for tandem mass spectrometry (MS/MS). The detector records the charge induced or current produced when an ion passes by or hits a surface. A mass spectrum is the result of measuring the signal produced in the detector when scanning m/z ions with a mass analyzer. Exemplary mass spectrometry analytical techniques include electrospray ionization mass spectroscopy (ESI), high resolution mass spectrometry (HRMS), liquid chromatography mass spectrometry (LCMS), and liquid chromatography-tandem mass spectrometry (LC-MS/MS). Also exemplary is MALDI mass spectrometry, such as MALDI-TOF mass spectrometry, wherein matrix-assisted laser desorption/ionization (MALDI) is the ion source, and the mass analyzer is time-of-flight (TOF) mass spectrometer.

C. Inhibitors of Ubiquitin-Like with PHD and Ring Finger Domains 1 (UHRF1)

In some embodiments, an inhibitor of UHRF1 is an antibody. The term “antibody” is used herein in the broadest sense and covers fully assembled antibodies, antibody fragments that retain the ability to specifically bind to the antigen (e.g., Fab, F(ab′)₂, Fv, and other fragments), single chain antibodies, diabodies, antibody chimeras, hybrid antibodies, bispecific antibodies, humanized antibodies, and the like.

In some embodiments, an inhibitor of UHRF1 is a small molecule. The term “small molecule” is used herein in the broadest sense and covers a molecule of less than 1,000 daltons, such as synthetic inorganic, organometallic, and organic molecule.

In some embodiments, an inhibitor of UHRF1 is an aptamer. The term “aptamer” as used herein is an oligonucleotide or a peptide molecule that specifically binds to a target. In some embodiments, the aptamer is an oligonucleotide having, e.g., about 15 to about 100 nucleotides, such as about 15 to about 50 nucleotides.

In some embodiments, an inhibitor of UHRF1 is a small interfering RNA (siRNA). The term “siRNA” or “small interfering RNA” or “short interfering RNA” or “silencing RNA” as used herein refers to a single- or double-stranded non-coding RNA, from about 20 to about 27 bases or base pairs in length, which interferes with expression of specific RNAs having complementary nucleotide sequences. In some embodiments an siRNA causes degradation of a mRNA, preventing translation of the protein product.

In some embodiments, an inhibitor of UHRF1 is an antisense oligonucleotide. The term “antisense oligonucleotide” as used herein refers to a single stranded oligonucleotide that is complementary to a particular sequence in a target gene or RNA, and modulates its expression or splicing. In some embodiments, the antisense oligonucleotide is at least 10, such as at least 15 nucleotides, and optionally between about 15 to about 30, such as about 15 to about 25 nucleotides, and may contain one or more modifications compared to naturally-occurring nucleotides.

In some embodiments, the specific amount of the inhibitor of UHRF1 provided herein for use in the methods provided herein is determined by factors such as the specific type of inhibitor used, the type of multiple myeloma being treated or managed, the severity and stage of disease, the age, height, and/or weight of the subject being treated; and any optional additional active agents concurrently administered to the patient.

### D. Methods of Use

In some embodiments, provided herein is a method of treating multiple myeloma, comprising administering to a subject with multiple myeloma an inhibitor of UHRF1.

In some embodiments, provided herein is a method of treating multiple myeloma, comprising administering to a subject with multiple myeloma a therapeutically effective amount of an inhibitor of UHRF1.

In some embodiments, the multiple myeloma is plasma cell leukemia (PCL).

In some embodiments, the multiple myeloma is newly diagnosed multiple myeloma.

In some embodiments, the multiple myeloma is relapsed or refractory. In some embodiments, the multiple myeloma is refractory to lenalidomide. In some embodiment, the multiple myeloma is refractory to pomalidomide. In some embodiments, the multiple myeloma is refractory to the combination of pomalidomide and a proteasome inhibitor. In some embodiments, the proteasome inhibitor is selected from bortezomib, carfilzomib, and ixazomib. In some embodiments, the multiple myeloma is refractory to the combination of pomalidomide and an inflammatory steroid. In some embodiments, the inflammatory steroid is selected from dexamethasone or prednisone. In some embodiments, the multiple myeloma is refractory to the combination of pomalidomide and an anti-CD38 antibody.

In some embodiment, provided herein are methods for achieving a complete response, partial response, or stable disease in a patient, comprising administering to subject with multiple myeloma an inhibitor of UHRF1.

In some embodiments, also provided herein are methods for inducing a therapeutic response assessed with the International Uniform Response Criteria for Multiple Myeloma (IURC) (see Durie BGM, Harousseau J-L, Miguel J S, et al. International uniform response criteria for multiple myeloma. Leukemia, 2006; (10) 10:1 7) of a patient, comprising administering to a subject with multiple myeloma an inhibitor of UHRF1.

In some embodiments, provided herein are methods for achieving a stringent complete response, complete response, or very good partial response, as determined by the International Uniform Response Criteria for Multiple Myeloma (IURC) in a patient, comprising administering to a subject with multiple myeloma an inhibitor of UHRF1.

In some embodiments, provided herein are methods for achieving an increase in overall survival, progression-free survival, event-free survival, time to progression, or disease-free survival in a patient, comprising administering to a subject with multiple myeloma an inhibitor of UHRF1.

Also provided herein, in some embodiments, is a method of selecting a subject with multiple myeloma for treatment with an UHRF1 inhibitor, comprising determining the level of UHRF1 in a sample obtained from the subject, wherein if the level of UHRF1 in the sample is above a reference level, the subject is selected for treatment with a UHRF1 inhibitor.

In some embodiments, provided herein is a method of selecting a subject with multiple myeloma for treatment with an UHRF1 inhibitor, comprising:
a) obtaining a sample from the subject;
b) determining whether the level of UHRF1 in the sample is above a reference level;
c) if the level of UHRF1 in the sample is above a reference level, selecting the subject for treatment with an UHRF1 inhibitor.

In some embodiments, provided herein is a method of predicting whether a subject with multiple myeloma will benefit from treatment with an UHRF1 inhibitor, comprising determining whether the level of UHRF1 in the sample from the subject is above a reference level, wherein if the level of UHRF1 in the sample is above a reference level, the subject is predicted to benefit from treatment with an NSD2 inhibitor.

In some embodiments, provided herein is a method of predicting whether a subject with multiple myeloma will benefit from treatment with an UHRF1 inhibitor, comprising:
a) obtaining a sample from the subject;
b) determining whether the level of UHRF1 in the sample is above a reference level;
c) if the level of UHRF1 in the sample is above a reference level, selecting the subject for treatment with an UHRF1 inhibitor.

In some embodiments, the sample is a blood sample, plasma sample, or cancer sample, such as a multiple myeloma sample.

Also provided herein are methods of treating patients who have been previously treated for multiple myeloma but are non-responsive to standard therapies, as well as those who have not previously been treated. Further encompassed are methods of treating patients who have undergone surgery in an attempt to treat multiple myeloma, as well as those who have not. Also provided herein are methods of treating patients who have been previously undergone transplant therapy, as well as those who have not.

The methods provided herein include treatment of multiple myeloma that is relapsed, refractory or resistant. The methods provided herein include prevention of multiple myeloma that is relapsed, refractory or resistant. The methods provided herein include management of multiple myeloma that is relapsed, refractory or resistant. In some such embodiments, the myeloma is primary, secondary, tertiary, quadruply, or quintuply relapsed multiple myeloma. In some embodiments, the methods provided herein reduce, maintain or eliminate minimal residual disease (MRD). In some embodiments, provided herein is a method of increasing rate and/or durability of MRD negativity in multiple myeloma patients, comprising administering to a subject with multiple myeloma an inhibitor of UHRF1. In some embodiments, methods provided herein encompass treating, preventing or managing various types of multiple myeloma, such as monoclonal gammopathy of undetermined significance (MGUS), low risk, intermediate risk, and high risk multiple myeloma, newly diagnosed multiple myeloma (including low risk, intermediate risk, and high risk newly diagnosed multiple myeloma), transplant eligible and transplant ineligible multiple myeloma, smoldering (indolent) multiple myeloma (including low risk, intermediate risk, and high risk smouldering multiple myeloma), active multiple myeloma, solitary plasmacytoma, extramedullary plasmacytoma, plasma cell leukemia, central nervous system multiple myeloma, light chain myeloma, non-secretory myeloma, Immunoglobulin D myeloma, and Immunoglobulin E myeloma, by administering to a subject with multiple myeloma an inhibitor of UHRF1.

In another embodiment, methods provided herein encompass treating, preventing or managing multiple myeloma characterized by genetic abnormalities, such as Cyclin D translocations (for example, t (11;14) (q13;q32); t (6;14) (p21;32); t (12;14) (p13;q32); or t (6;20);); MMSET translocations (for example, t (4;14) (p16;q32)); MAF translocations (for example, t (14;16) (q32;q32); t (20;22); t (16; 22) (q11;q13); or t (14;20) (q32;q11)); or other chromosome factors (for example, deletion of 17p13, or chromosome 13; del (17/17p), nonhyperdiploidy, and gain (1q)), by administering an inhibitor of UHRF1. In some embodiments, the multiple myeloma is characterized according to the multiple myeloma International Staging System (ISS). In some embodiments, the multiple myeloma is Stage I multiple myeloma as characterized by ISS (e.g., serum β2 microglobulin <3.5 mg/L and serum albumin ≥3.5 g/dL). In some embodiments, the multiple myeloma is Stage III multiple myeloma as characterized by ISS (e.g., serum β2 microglobulin >5.4 mg/L). In one embodiment, the multiple myeloma is Stage II multiple myeloma as characterized by ISS (e.g., not Stage I or III).

In some embodiments, the methods comprise administering an inhibitor of UHRF1. In some embodiments, the methods comprise administering an inhibitor of UHRF1 as consolidation therapy. In some embodiments, the methods comprise administering an inhibitor of UHRF1 as maintenance therapy.

In one particular embodiment of the methods described herein, the multiple myeloma is plasma cell leukemia.

In some embodiments, the multiple myeloma is high risk multiple myeloma. In some such embodiments, the high risk multiple myeloma is relapsed or refractory. In some embodiments, the high risk multiple myeloma is multiple myeloma that is relapsed within 12 months of first treatment. In some embodiments, the high risk multiple myeloma is multiple myeloma that is additionally characterized by genetic abnormalities, for example, one or more of del (17/17p) and t (14;16) (q32;q32). In some such embodiments, the high risk multiple myeloma is relapsed or refractory to one, two or three previous treatments.

In some embodiments, the multiple myeloma is additionally characterized by a p53 mutation. In some embodiments, the p53 mutation is a Q331 mutation. In some embodiments, the p53 mutation is an R273H mutation. In some embodiments, the p53 mutation is a K132 mutation. In some embodiments, the p53 mutation is a K132N mutation. In some embodiments, the p53 mutation is an R337 mutation. In some embodiments, the p53 mutation is an R337L mutation. In some embodiments, the p53 mutation is a W146 mutation. In some embodiments, the p53 mutation is an S261 mutation. In some embodiments, the p53 mutation is an S261T mutation. In some embodiments, the p53 mutation is an E286 mutation. In some embodiments, the p53 mutation is an E286K mutation. In some embodiments, the p53 mutation is an R175 mutation. In some embodiments, the p53 mutation is an R175H mutation. In some embodiments, the p53 mutation is an E258 mutation. In some embodiments, the p53 mutation is an E258K mutation. In some embodiments, the p53 mutation is an A161 mutation. In some embodiments, the p53 mutation is an A161T mutation.

In some embodiments, the multiple myeloma is characterized by homozygous deletion of p53. In some embodiments, the multiple myeloma is characterized by homozygous deletion of wild type p53.

In some embodiments, the multiple myeloma is characterized by wild type p53.

In some embodiments, the multiple myeloma is characterized by activation of one or more oncogenic drivers. In some embodiments, the one or more oncogenic drivers are selected from the group consisting of C-MAF, MAFB, FGFR3, MMset, Cyclin D1, and Cyclin D. In some embodiments, the multiple myeloma is characterized by activation of C MAF. In some embodiments, the multiple myeloma is characterized by activation of MAFB. In some embodiments, the multiple myeloma is characterized by activation of FGFR3 and MMset. In some embodiments, the multiple myeloma is characterized by activation of C MAF, FGFR3, and MMset. In certain embodiments, the multiple myeloma is characterized by activation of Cyclin D1. In some embodiments, the multiple myeloma is characterized by activation of MAFB and Cyclin D1. In some embodiments, the multiple myeloma is characterized by activation of Cyclin D.

In some embodiments, the multiple myeloma is characterized by one or more chromosomal translocations, such as one or more of t (14;16), t (14;20), t (11;14), t (6;20), t (20;22), and t (16;22).

In some embodiments, the multiple myeloma is characterized by a Q331 p53 mutation, by activation of C-MAF, and by chromosomal translocation t (14;16). In some embodiments, the multiple myeloma is characterized by homozygous deletion of p53, by activation of C-MAF, and by chromosomal translocation at t (14;16). In some embodiments, the multiple myeloma is characterized by a K132N p53 mutation, by activation of MAFB, and by a chromosomal translocation at t (14;20). In some embodiments, the multiple myeloma is characterized by wild type p53 and by activation of FGFR3 and MMset. In some embodiments, the multiple myeloma is characterized by wild type p53, by activation of C-MAF, and by chromosomal translocation at t (14;16). In some embodiments, the multiple myeloma is characterized by homozygous deletion of p53, by activation of FGFR3, MMset, and C MAF, and by chromosomal translocation at t (14;16). In some embodiments, the multiple myeloma is characterized by homozygous deletion of p53, by activation of Cyclin D1, and by chromosomal translocation at t (11;14). In some embodiments, the multiple myeloma is characterized by an R337L p53 mutation, by activation of Cyclin D1, and by chromosomal translocation at t (11;14). In some embodiments, the multiple myeloma is characterized by a W146 p53 mutation, by activation of FGFR3 and MMset, and by chromosomal translocation at t (4;14). In some embodiments, the multiple myeloma is characterized by an S261T p53 mutation, by activation of MAFB, and by chromosomal translocations at t (6;20) and t (20;22). In some embodiments, the multiple myeloma is characterized by an E286K p53 mutation, by activation of FGFR3 and MMset, and by a chromosomal translocation at t (4;14). In some embodiments, the multiple myeloma is characterized by an R175H p53 mutation, by activation of FGFR3 and MMset, and by a chromosomal translocation at t (4;14). In some embodiments, the multiple myeloma is characterized by an E258K p53 mutation, by activation of C-MAF, and by chromosomal translocations at t (14;16) and t (16;22). In some embodiments, the multiple myeloma is characterized by wild type p53, by activation of MAFB and Cyclin D1, and by chromosomal translocations at t (14;20) and t (11;14). In some embodiments, the multiple myeloma is characterized by an A161T p53 mutation, by activation of Cyclin D, and by chromosomal translocation at t (11;14).

In some embodiments of the methods described herein, the multiple myeloma is transplant eligible newly diagnosed multiple myeloma. In another embodiment, the multiple myeloma is transplant ineligible newly diagnosed multiple myeloma.

In yet other embodiments, the multiple myeloma is characterized by early progression (for example less than 12 months) following initial treatment. In still other embodiments, the multiple myeloma is characterized by early progression (for example less than 12 months) following autologous stem cell transplant. In some embodiments, the multiple myeloma is refractory to lenalidomide. In some embodiments, the multiple myeloma is refractory to pomalidomide. In some such embodiments, the multiple myeloma is predicted to be refractory to pomalidomide (for example, by molecular characterization). In some embodiments, the multiple myeloma is relapsed or refractory to 3 or more treatments and was exposed to a proteasome inhibitor (for example, bortezomib, carfilzomib, ixazomib, oprozomib, or marizomib) and an immunomodulatory compound (for example thalidomide, lenalidomide, pomalidomide, iberdomide, or avadomide), or double refractory to a proteasome inhibitor and an immunomodulatory compound. In still other embodiments, the multiple myeloma is relapsed or refractory to 3 or more prior therapies, including for example, a CD38 monoclonal antibody (CD38 mAb, for example, daratumumab or isatuximab), a proteasome inhibitor (for example, bortezomib, carfilzomib, ixazomib, or marizomib), and an immunomodulatory compound (for example thalidomide, lenalidomide, pomalidomide, iberdomide, or avadomide) or double refractory to a proteasome inhibitor or immunomodulatory compound and a CD38 mAb. In still other embodiments, the multiple myeloma is triple refractory, for example, the multiple myeloma is refractory to a proteasome inhibitor (for example, bortezomib, carfilzomib, ixazomib, oprozomib or marizomib), an immunomodulatory compound (for example thalidomide, lenalidomide, pomalidomide, iberdomide, or avadomide), and one other active agent, as described herein.

In certain embodiments, provided herein are methods of treating, preventing, and/or managing multiple myeloma, including relapsed/refractory multiple myeloma in a subject with impaired renal function or a symptom thereof, comprising administering an inhibitor of UHRF1, to a subject having relapsed/refractory multiple myeloma with impaired renal function.

In certain embodiments, provided herein are methods of treating, preventing, and/or managing multiple myeloma, including relapsed or refractory multiple myeloma in a frail subject, comprising administering an inhibitor of UHRF1, to a frail subject having multiple myeloma. In some such embodiments, the frail subject is characterized by ineligibility for induction therapy, or intolerance to dexamethasone treatment. In some such embodiment the frail subject is elderly, for example, older than 65 years old.

In certain embodiments, provided herein are methods of treating, preventing or managing multiple myeloma, comprising administering to a subject an inhibitor of UHRF1, wherein the multiple myeloma is fourth line relapsed/refractory multiple myeloma.

In certain embodiments, provided herein are methods of treating, preventing or managing multiple myeloma, comprising administering to a subject an inhibitor of UHRF1, as induction therapy, wherein the multiple myeloma is newly diagnosed, transplant-eligible multiple myeloma.

In certain embodiments, provided herein are methods of treating, preventing or managing multiple myeloma, comprising administering to a subject an inhibitor of UHRF1, as maintenance therapy after other therapy or transplant, wherein the multiple myeloma is newly diagnosed, transplant-eligible multiple myeloma prior to the other therapy or transplant.

In certain embodiments, provided herein are methods of treating, preventing or managing multiple myeloma, comprising administering to a subject an inhibitor of UHRF1, as maintenance therapy after other therapy or transplant. In some embodiments, the multiple myeloma is newly diagnosed, transplant-eligible multiple myeloma prior to the other therapy and/or transplant. In some embodiments, the other therapy prior to transplant is treatment with chemotherapy or an inhibitor of UHRF1.

In certain embodiments, provided herein are methods of treating, preventing or managing multiple myeloma, comprising administering to a subject an inhibitor of UHRF1, wherein the multiple myeloma is high risk multiple myeloma, that is relapsed or refractory to one, two or three previous treatments.

In certain embodiments, provided herein are methods of treating, preventing or managing multiple myeloma, comprising administering to a subject an inhibitor of UHRF1, wherein the multiple myeloma is newly diagnosed, transplant-ineligible multiple myeloma.

In certain embodiments, the subject to be treated with one of the methods provided herein has not been treated with multiple myeloma therapy prior to the administration of an inhibitor of UHRF1. In certain embodiments, the subject to be treated with one of the methods provided herein has been treated with multiple myeloma therapy prior to the administration of an inhibitor of UHRF1. In certain embodiments, the subject to be treated with one of the methods provided herein has developed drug resistance to the anti-multiple myeloma therapy. In some such embodiments, the subject has developed resistance to one, two, or three anti-multiple myeloma therapies, wherein the therapies are selected from a CD38 monoclonal antibody (CD38 mAb, for example, daratumumab or isatuximab), a proteasome inhibitor (for example, bortezomib, carfilzomib, ixazomib, or marizomib), and an immunomodulatory compound (for example thalidomide, lenalidomide, pomalidomide, iberdomide, or avadomide).

The methods provided herein encompass treating a subject regardless of patient's age. In some embodiments, the subject is 18 years or older. In other embodiments, the subject is more than 18, 25, 35, 40, 45, 50, 55, 60, 65, or 70 years old. In other embodiments, the subject is less than 65 years old. In other embodiments, the subject is more than 65 years old. In some embodiments, the subject is an elderly multiple myeloma subject, such as a subject older than 65 years old. In some embodiments, the subject is an elderly multiple myeloma subject, such as a subject older than 75 years old.

E. Combination Therapy with At Least One Second Therapeutic Agent

In some embodiments, the methods provided herein (use of an inhibitor of UHRF1) additionally comprises administering to the subject at least one second therapeutic agent, also referred to herein as an “additional agent” or “additional active agent.”

In some embodiments, the at least one second therapeutic agent is selected from a chemotherapy agent, a steroid, an immunomodulating agent, a proteasome inhibitor, a histone deacetylase inhibitor, an anti-CD38 antibody, and anti-SLAMF7 antibody, an antibody-drug conjugate, and a nuclear export inhibitor.

In some embodiments, the at least one second therapeutic agent is selected from lenalidomide, thalidomide, pomalidomide, iberdomide, avadomide, bortezomib, carfilzomib, ixazomib, panobinostat, melphalan, vincristine, cyclophosphamide, etoposide, doxorubicin, and bendamustine, dexamethasone, prednisone, daratumumab, isatuximab, elotuzumab, belantamab mafodotin-blmf, selinexor, pamidronate, zoledronic acid, and denosumab.

In some embodiments, the at least one second therapeutic agent is lenalidomide. In some embodiments, the at least one second therapeutic agent is iberdomide. In some embodiments, the at least one second therapeutic agent is(S)-4-(4-(4-(((2-(2,6-dioxopiperidin-3-yl)-1-oxoisoindolin-4-yl)oxy)methyl)benzyl) piperazin-1-yl)-3-fluorobenzonitrile. In some embodiments, the at least one second therapeutic agent is (i) lenalidomide, pomalidomide, or thalidomide; and (ii) dexamethasone. In some embodiments, the at least one second therapeutic agent is (i) carfilzomib, ixazomib, or bortezomib; (ii) lenalidomide; and (iii) dexamethasone. In some embodiments, the at least one second therapeutic agent is (i) bortezomib or carfilzomib; (ii) cyclophosphamide; and (iii) dexamethasone. In some embodiments, the at least one second therapeutic agent is (i) elotuzumab or daratumumab; (ii) lenalidomide; and (iii) dexamethasone. In some embodiments, the at least one second therapeutic agent is bortezomib, liposomal doxorubicin, and dexamethasone. In some embodiments, the at least one second therapeutic agent is panobinostat, bortezomib, and dexamethasone. In some embodiments, the at least one second therapeutic agent is elotuzumab, bortezomib, and dexamethasone. In some embodiments, the at least one second therapeutic agent is melphalan and prednisone, with or without thalidomide or bortezomib. In some embodiments, the at least one second therapeutic agent is vincristine, doxorubicin, and dexamethasone. In some embodiments, the at least one second therapeutic agent is dexamethasone, cyclophosphamide, etoposide, and cisplatin. In some embodiments, the at least one second therapeutic agent is dexamethasone, thalidomide, cisplatin, doxorubicin, cyclophosphamide, and etoposide, with or without bortezomib.

In some embodiments, the at least one second therapeutic agent is a steroid.

In some embodiments, the specific amount (dosage) of the at least one second therapeutic agent provided herein as used in the methods provided herein is determined by factors such as the specific agent used, the type of multiple myeloma being treated or managed, the severity and stage of disease, the amount of an inhibitor of UHRF1 provided herein, and any optional additional active agents concurrently administered to the patient.

In some embodiments, the dosage of an at least one second therapeutic agent provided herein as used in the methods provided herein is determined based on a commercial package insert of medicament (e.g., a label) as approved by the FDA or a similar regulatory agency of a country other than the USA for said active agent. In some embodiments, the dosage of a second therapeutic agent provided herein as used in the methods provided herein is a dosage approved by the FDA or a similar regulatory agency of a country other than the USA for said therapeutic agent. In some embodiments, the dosage of a second therapeutic agent provided herein as used in the methods provided herein is a dosage used in a human clinical trial for said therapeutic agent. In some embodiments, the dosage of a second therapeutic agent provided herein as used in the methods provided herein is lower than a dosage approved by the FDA or a similar regulatory agency of a country other than the USA for said therapeutic agent or a dosage used in a human clinical trial for said active agent, depending on, e.g., the synergistic effects between the second therapeutic agent and an inhibitor of UHRF1 as provided herein.

The combined use of an inhibitor of UHRF1 provided herein can also be combined or used in conjunction with (e.g. before, during, or after) conventional therapy including, but not limited to, surgery, biological therapy (including immunotherapy, for example with checkpoint inhibitors), radiation therapy, chemotherapy, stem cell transplantation, cell therapy, or other non-drug based therapy presently used to treat, prevent or manage cancer (e.g., multiple myeloma). The combined use of an inhibitor of UHRF1 provided herein and conventional therapy may provide a unique treatment regimen that is unexpectedly effective in certain patients. Without being limited by theory, it is believed that an inhibitor of UHRF1 provided herein may provide additive or synergistic effects when given concurrently with conventional therapy.

As discussed elsewhere herein, encompassed herein is a method of reducing, treating and/or preventing adverse or undesired effects associated with conventional therapy including, but not limited to, surgery, chemotherapy, radiation therapy, biological therapy and immunotherapy. An inhibitor of UHRF1 provided herein, and an at least one second therapeutic agent ingredient can be administered to a patient prior to, during, or after the occurrence of the adverse effect associated with conventional therapy. In some embodiments, the at least one second therapeutic agent is dexamethasone.

The inhibitor of UHRF1 provided herein can also be further combined or used in combination with other therapeutic agents useful in the treatment and/or prevention of multiple myeloma described herein. In one such embodiment, the at least one second therapeutic agent is dexamethasone.

In some embodiments, provided herein is a method of treating, preventing, or managing multiple myeloma, comprising administering to a patient an inhibitor of UHRF1 provided herein, further in combination with one or more additional therapeutic agents, and optionally further in combination with radiation therapy, blood transfusions, or surgery.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. A first therapy (e.g., a prophylactic or therapeutic agent such as an inhibitor of UHRF1 provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., an at least one second therapeutic agent) to the subject. The first therapy and the second therapy independently can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a third therapy (e.g., an additional prophylactic or therapeutic agent) to the subject. Quadruple therapy is also contemplated herein, as is quintuple therapy. In some embodiments, the second therapy is dexamethasone.

Administration of an inhibitor of UHRF1 provided herein, and one or more second therapeutic agents to a subject can occur simultaneously or sequentially by the same or different routes of administration. The suitability of a particular route of administration employed for a particular therapeutic agent will depend on the active agent itself (e.g., whether it can be administered orally without decomposing prior to entering the blood stream).

The route of administration of an inhibitor of UHRF1 provided herein is independent of the additional therapy. In some embodiments, an inhibitor of UHRF1 provided herein is administered orally. In another embodiment, an inhibitor of UHRF1 provided herein is administered intravenously. Thus, in accordance with these embodiments, an inhibitor of UHRF1 provided herein is administered orally or intravenously, and the additional therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In some embodiments, an inhibitor of UHRF1 provided herein, and an additional therapy are administered by the same mode of administration, orally or by IV. In another embodiment, an inhibitor of UHRF1 provided herein is administered by one mode of administration, e.g., by IV, whereas the additional agent (an anti-multiple myeloma agent) is administered by another mode of administration, e.g., orally.

In some embodiments, the at least one second therapeutic agent is administered intravenously or subcutaneously and once or twice daily in an amount of from about 1 to about 1000 mg, from about 5 to about 500 mg, from about 10 to about 350 mg, or from about 50 to about 200 mg. The specific amount of the additional active agent will depend on the specific agent used, the type of multiple myeloma being treated or managed, the severity and stage of disease, the amount of inhibitor of UHRF1 provided herein, and any optional additional active agents concurrently administered to the subject.

One or more additional active ingredients or agents can be used together with an inhibitor of UHRF1 provided herein in the methods and compositions provided herein. Additional active agents can be large molecules (e.g., proteins), small molecules (e.g., synthetic inorganic, organometallic, or organic molecules), or cell therapies (e.g., CAR cells).

Examples of additional active agents that can be used in the methods and compositions described herein include one or more of melphalan, vincristine, cyclophosphamide, etoposide, doxorubicin, bendamustine, obinutuzmab, a proteasome inhibitor (for example, bortezomib, carfilzomib, ixazomib, oprozomib or marizomib), a deacetylase inhibitor, such as a histone deacetylase inhibitor (as described herein, for example, panobinostat, ACY241), a BET inhibitor (for example, GSK525762A, OTX015, BMS-986158, TEN-010, CPI-0610, INCB54329, BAY1238097, FT-1101, ABBV-075, BI 894999, GS-5829, GSK1210151A (I-BET-151), CPI-203, RVX-208, XD46, MS436, PFI-1, RVX2135, ZEN3365, XD14, ARV-771, MZ-1, PLX5117, 4-[2-(cyclopropylmethoxy)-5-(methanesulfonyl)phenyl]-2-methylisoquinolin-1 (2H)-one, EP11313 and EP11336), a BCL2 inhibitor (for example, venetoclax or navitoclax), an MCL-1 inhibitor (for example, AZD5991, AMG176, MIK665, S64315, or S63845), an LSD-1 inhibitor (for example, ORY-1001, ORY-2001, INCB-59872, IMG-7289, TAK-418, GSK-2879552, 4-[2-(4-amino-piperidin-1-yl)-5-(3-fluoro-4-methoxy-phenyl)-1-methyl-6-oxo-1,6-dihydropyrimidin-4-yl]-2-fluoro-benzonitrile or a salt thereof), a corticosteroid (for example, prednisone), dexamethasone; an antibody (for example, a CS1 antibody, such as elotuzumab; a CD38 antibody, such as daratumumab or isatuximab; or a BCMA antibody or antibody-conjugate, such as GSK2857916 or BI 836909), a checkpoint inhibitor (as described herein), or CAR cells (as described herein); or an anti-SLAMF7 antibody (as described herein); or an antibody-drug conjugate (as described herein); or a nuclear export inhibitor (as described herein).

In some embodiments, the additional active agent used together with an inhibitor of UHRF1 provided herein, in the methods and compositions described herein is dexamethasone.

In some embodiments, the dexamethasone is administered at a 4 mg dose on days 1 and 8 of a 21 day cycle. In some other embodiments, the dexamethasone is administered at a 4 mg dose on days 1, 4, 8 and 11 of a 21 day cycle. In some embodiments, the dexamethasone is administered at a 4 mg dose on days 1, 8, and 15 of a 28 day cycle. In some other embodiments, the dexamethasone is administered at a 4 mg dose on days 1, 4, 8, 11, 15 and 18 of a 28 day cycle. In some embodiments, the dexamethasone is administered at a 4 mg dose on days 1, 8, 15, and 22 of a 28 day cycle. In one such embodiment, the dexamethasone is administered at a 4 mg dose on days 1, 10, 15, and 22 of Cycle 1. In some embodiments, the dexamethasone is administered at a 4 mg dose on days 1, 3, 15, and 17 of a 28 day cycle. In one such embodiment, the dexamethasone is administered at a 4 mg dose on days 1, 3, 14, and 17 of Cycle 1.

In some other embodiments, the dexamethasone is administered at an 8 mg dose on days 1 and 8 of a 21 day cycle. In some other embodiments, the dexamethasone is administered at an 8 mg dose on days 1, 4, 8 and 11 of a 21 day cycle. In some embodiments, the dexamethasone is administered at an 8 mg dose on days 1, 8, and 15 of a 28 day cycle. In some other embodiments, the dexamethasone is administered at an 8 mg dose on days 1, 4, 8, 11, 15 and 18 of a 28 day cycle. In some embodiments, the dexamethasone is administered at an 8 mg dose on days 1, 8, 15, and 22 of a 28 day cycle. In one such embodiment, the dexamethasone is administered at an 8 mg dose on days 1, 10, 15, and 22 of Cycle 1. In some embodiments, the dexamethasone is administered at an 8 mg dose on days 1, 3, 15, and 17 of a 28 day cycle. In one such embodiment, the dexamethasone is administered at an 8 mg dose on days 1, 3, 14, and 17 of Cycle 1.

In some embodiments, the dexamethasone is administered at a 10 mg dose on days 1 and 8 of a 21 day cycle. In some other embodiments, the dexamethasone is administered at a 10 mg dose on days 1, 4, 8 and 11 of a 21 day cycle. In some embodiments, the dexamethasone is administered at a 10 mg dose on days 1, 8, and 15 of a 28 day cycle. In some other embodiments, the dexamethasone is administered at a 10 mg dose on days 1, 4, 8, 11, 15 and 18 of a 28 day cycle. In some embodiments, the dexamethasone is administered at a 10 mg dose on days 1, 8, 15, and 22 of a 28 day cycle. In some embodiments, the dexamethasone is administered at a 10 mg dose on days 1, 10, 15, and 22 of Cycle 1. In some embodiments, the dexamethasone is administered at a 10 mg dose on days 1, 3, 15, and 17 of a 28 day cycle. In some embodiments, the dexamethasone is administered at a 10 mg dose on days 1, 3, 14, and 17 of Cycle 1.

In some embodiments, the dexamethasone is administered at a 20 mg dose on days 1 and 8 of a 21 day cycle. In some other embodiments, the dexamethasone is administered at a 20 mg dose on days 1, 4, 8 and 11 of a 21 day cycle. In some embodiments, the dexamethasone is administered at a 20 mg dose on days 1, 8, and 15 of a 28 day cycle. In some other embodiments, the dexamethasone is administered at a 20 mg dose on days 1, 4, 8, 11, 15 and 18 of a 28 day cycle. In some embodiments, the dexamethasone is administered at a 20 mg dose on days 1, 8, 15, and 22 of a 28 day cycle. In some embodiments, the dexamethasone is administered at a 20 mg dose on days 1, 10, 15, and 22 of Cycle 1. In some embodiments, the dexamethasone is administered at a 20 mg dose on days 1, 3, 15, and 17 of a 28 day cycle. In some embodiments, the dexamethasone is administered at a 20 mg dose on days 1, 3, 14, and 17 of Cycle 1.

In some embodiments, the dexamethasone is administered at a 40 mg dose on days 1 and 8 of a 21 day cycle. In some other embodiments, the dexamethasone is administered at a 40 mg dose on days 1, 4, 8 and 11 of a 21 day cycle. In some embodiments, the dexamethasone is administered at a 40 mg dose on days 1, 8, and 15 of a 28 day cycle. In one such embodiment, the dexamethasone is administered at a 40 mg dose on days 1, 10, 15, and 22 of Cycle 1. In some other embodiments, the dexamethasone is administered at a 40 mg dose on days 1, 4, 8, 11, 15 and 18 of a 28 day cycle. In other such embodiments, the dexamethasone is administered at a 40 mg dose on days 1, 8, 15, and 22 of a 28 day cycle. In other such embodiments, the dexamethasone is administered at a 40 mg dose on days 1, 3, 15, and 17 of a 28 day cycle. In some embodiments, the dexamethasone is administered at a 40 mg dose on days 1, 3, 14, and 17 of Cycle 1.

In another embodiment, the additional active agent used together with an inhibitor of UHRF1 provided herein in the methods and compositions described herein is bortezomib. In yet another embodiment, the additional active agent used together with an inhibitor of UHRF1 provided herein in the methods and compositions described herein is daratumumab. In some such embodiments, the methods additionally comprise administration of dexamethasone. In some embodiments, the methods comprise administration of an inhibitor of UHRF1 provided herein with a proteasome inhibitor as described herein, a CD38 inhibitor as described herein and a corticosteroid as described herein.

In certain embodiments, an inhibitor of UHRF1 provided herein is administered in combination with checkpoint inhibitors. In some embodiments, one checkpoint inhibitor is used in combination with an inhibitor of UHRF1 provided herein in connection with the methods provided herein. In another embodiment, two checkpoint inhibitors are used in combination with an inhibitor of UHRF1 provided herein in connection with the methods provided herein. In yet another embodiment, three or more checkpoint inhibitors are used in combination with an inhibitor of UHRF1 provided herein in connection with the methods provided herein.

As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In some embodiments, the checkpoint inhibitor is a CTLA-4 inhibitor. In some embodiments, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In some embodiments, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In some embodiments, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In some embodiments, the checkpoint inhibitor is a PD-1 inhibitor. In some embodiments, the PD-1 inhibitor is an anti-PD-1 antibody. In some embodiments, the anti-PD-1 antibody is BGB-A317, nivolumab (also known as ONO-4538, BMS-936558, or MDX1106) or pembrolizumab (also known as MK-3475, SCH 900475, or lambrolizumab). In some embodiments, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In some embodiments, the checkpoint inhibitor is a PD-L1 inhibitor. In some embodiments, the PD-L1 inhibitor is an anti-PD-L1 antibody. In some embodiments, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In some embodiments, the checkpoint inhibitor is a PD-L2 inhibitor. In some embodiments, the PD-L2 inhibitor is an anti-PD-L2 antibody. In some embodiments, the anti-PD-L2 antibody is rHIgM12B7A.

In some embodiments, the checkpoint inhibitor is a lymphocyte activation gene-3 (LAG-3) inhibitor. In some embodiments, the LAG-3 inhibitor is IMP321, a soluble Ig fusion protein (Brignone et al., J. Immunol., 2007, 179, 4202-4211). In another embodiment, the LAG-3 inhibitor is BMS-986016.

In some embodiments, the checkpoint inhibitors is a B7 inhibitor. In some embodiments, the B7 inhibitor is a B7-H3 inhibitor or a B7-H4 inhibitor. In some embodiments, the B7-H3 inhibitor is MGA271, an anti-B7-H3 antibody (Loo et al., Clin. Cancer Res., 2012, 3834).

In some embodiments, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In some embodiments, the checkpoint inhibitor is an OX40 (CD134) agonist. In some embodiments, the checkpoint inhibitor is an anti-OX40 antibody. In some embodiments, the anti-OX40 antibody is anti-OX-40. In another embodiment, the anti-OX40 antibody is MEDI6469.

In some embodiments, the checkpoint inhibitor is a GITR agonist. In some embodiments, the checkpoint inhibitor is an anti-GITR antibody. In some embodiments, the anti-GITR antibody is TRX518.

In some embodiments, the checkpoint inhibitor is a CD137 agonist. In some embodiments, the checkpoint inhibitor is an anti-CD137 antibody. In some embodiments, the anti-CD137 antibody is urelumab. In another embodiment, the anti-CD137 antibody is PF-05082566.

In some embodiments, the checkpoint inhibitor is a CD40 agonist. In some embodiments, the checkpoint inhibitor is an anti-CD40 antibody. In some embodiments, the anti-CD40 antibody is CF-870,893.

In some embodiment, the checkpoint inhibitor is recombinant human interleukin-15 (rhIL-15).

In some embodiment, the checkpoint inhibitor is an IDO inhibitor. In some embodiments, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with one or more second therapeutic agents as described herein where appropriate for treating diseases described herein and understood in the art.

In certain embodiments, an inhibitor of UHRF1 provided herein can be used in combination with one or more immune cells expressing one or more chimeric antigen receptors (CARs) on their surface (e.g., a modified immune cell). Generally, CARs comprise an extracellular domain from a first protein (e.g., an antigen-binding protein), a transmembrane domain, and an intracellular signaling domain. In certain embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein.

Extracellular domains: The extracellular domains of the CARs bind to an antigen of interest. In certain embodiments, the extracellular domain of the CAR comprises a receptor, or a portion of a receptor, that binds to said antigen. In certain embodiments, the extracellular domain comprises, or is, an antibody or an antigen-binding portion thereof. In specific embodiments, the extracellular domain comprises, or is, a single chain Fv (scFv) domain. The single-chain Fv domain can comprise, for example, a VL linked to VH by a flexible linker, wherein said V_{L} and V_{H} are from an antibody that binds said antigen.

In certain embodiments, the antigen recognized by the extracellular domain of a polypeptide described herein is a tumor-associated antigen (TAA) or a tumor-specific antigen (TSA). In various specific embodiments, the tumor-associated antigen or tumor-specific antigen is, without limitation, Her2, prostate stem cell antigen (PSCA), alpha-fetoprotein (AFP), carcinoembryonic antigen (CEA), cancer antigen-125 (CA-125), CA19-9, calretinin, MUC-1, B cell maturation antigen (BCMA), epithelial membrane protein (EMA), epithelial tumor antigen (ETA), tyrosinase, melanoma-24 associated antigen (MAGE), CD19, CD22, CD27, CD30, CD34, CD45, CD70, CD99, CD117, EGFRvIII (epidermal growth factor variant III), mesothelin, PAP (prostatic acid phosphatase), prostein, TARP (T cell receptor gamma alternate reading frame protein), Trp-p8, STEAPI (six-transmembrane epithelial antigen of the prostate 1), chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-I), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein. In certain other embodiments, the TAA or TSA recognized by the extracellular domain of a CAR is integrin αvβ3 (CD61), galactin, or Ral-B.

In certain embodiments, the TAA or TSA recognized by the extracellular domain of a CAR is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXBI, SPA17, SSX, SYCPI, or TPTE.

In certain other embodiments, the TAA or TSA recognized by the extracellular domain of a CAR is a carbohydrate or ganglioside, e.g., fuc-GMI, GM2 (oncofetal antigen-immunogenic-1; OFA-I-1); GD2 (OFA-I-2), GM3, GD3, and the like.

In certain other embodiments, the TAA or TSA recognized by the extracellular domain of a CAR is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-l, dek-can fusion protein, EBNA, EF2, Epstein Barr virus antigens, ETV6-AML 1 fusion protein, HLA-A2, HLA-All, hsp70-2, KIAA0205, Mart2, Mum-1, 2, and 3, neo-PAP, myosin class I, OS-9, pml-RARa fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel17), tyrosinase, TRP-1, TRP-2, MAGE-1, MAGE-3, RAGE, GAGE-1, GAGE-2, p15 (58), RAGE, SCP-1, Hom/Mel-40, PRAME, p53, HRas, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, or TPS.

In various specific embodiments, the tumor-associated antigen or tumor-specific antigen is an AML-related tumor antigens, as described in S. Anguille et al, Leukemia (2012), 26, 2186-2196.

Other tumor-associated and tumor-specific antigens are known to those in the art.

Receptors, antibodies, and scFvs that bind to TSAs and TAAs, useful in constructing chimeric antigen receptors, are known in the art, as are nucleotide sequences that encode them.

In certain specific embodiments, the antigen recognized by the extracellular domain of a chimeric antigen receptor is an antigen not generally considered to be a TSA or a TAA, but which is nevertheless associated with tumor cells, or damage caused by a tumor. In certain embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the antigen is a DAMP, e.g., a heat shock protein, chromatin-associated protein high mobility group box 1 (HMGB 1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

Transmembrane domain: In certain embodiments, the extracellular domain of the CAR is joined to the transmembrane domain of the polypeptide by a linker, spacer or hinge polypeptide sequence, e.g., a sequence from CD28 or a sequence from CTLA4. The transmembrane domain can be obtained or derived from the transmembrane domain of any transmembrane protein, and can include all or a portion of such transmembrane domain. In specific embodiments, the transmembrane domain can be obtained or derived from, e.g., CD8, CD16, a cytokine receptor, and interleukin receptor, or a growth factor receptor, or the like.

Intracellular signaling domains: In certain embodiments, the intracellular domain of a CAR is or comprises an intracellular domain or motif of a protein that is expressed on the surface of T cells and triggers activation and/or proliferation of said T cells. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD35) or ITAM-containing portions thereof. In a specific embodiment, the intracellular domain is a CD35 intracellular signaling domain. In other specific embodiments, the intracellular domain is from a lymphocyte receptor chain, a TCR/CD3 complex protein, an Fe receptor subunit or an IL-2 receptor subunit. In certain embodiments, the CAR additionally comprises one or more co-stimulatory domains or motifs, e.g., as part of the intracellular domain of the polypeptide. The one or more co-stimulatory domains or motifs can be, or can comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof.

The CAR may also comprise a T cell survival motif. The T cell survival motif can be any polypeptide sequence or motif that facilitates the survival of the T lymphocyte after stimulation by an antigen. In certain embodiments, the T cell survival motif is, or is derived from, CD3, CD28, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor.

The modified immune cells expressing the CARs can be, e.g., T lymphocytes (T cells, e.g., CD4+ T cells or CD8+ T cells), cytotoxic lymphocytes (CTLs) or natural killer (NK) cells. T lymphocytes used in the compositions and methods provided herein may be naive T lymphocytes or MHC-restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T cells have been isolated from, or are expanded from T lymphocytes isolated from, peripheral blood, cord blood, or lymph. Immune cells to be used to generate modified immune cells expressing a CAR can be isolated using art-accepted, routine methods, e.g., blood collection followed by apheresis and optionally antibody-mediated cell isolation or sorting.

The modified immune cells are preferably autologous to an individual to whom the modified immune cells are to be administered. In certain other embodiments, the modified immune cells are allogeneic to an individual to whom the modified immune cells are to be administered. Where allogeneic T lymphocytes or NK cells are used to prepare modified T lymphocytes, it is preferable to select T lymphocytes or NK cells that will reduce the possibility of graft-versus-host disease (GVHD) in the individual. For example, in certain embodiments, virus-specific T lymphocytes are selected for preparation of modified T lymphocytes; such lymphocytes will be expected to have a greatly reduced native capacity to bind to, and thus become activated by, any recipient antigens. In certain embodiments, recipient-mediated rejection of allogeneic T lymphocytes can be reduced by co-administration to the host of one or more immunosuppressive agents, e.g., cyclosporine, tacrolimus, sirolimus, cyclophosphamide, or the like.

T lymphocytes, e.g., unmodified T lymphocytes, or T lymphocytes expressing CD3 and CD28, or comprising a polypeptide comprising a CD3 signaling domain and a CD28 co-stimulatory domain, can be expanded using antibodies to CD3 and CD28, e.g., antibodies attached to beads; see, e.g., U.S. Pat. Nos. 5,948,893; 6,534,055; 6,352,694; 6,692,964; 6,887,466; and 6,905,681.

The modified immune cells, e.g., modified T lymphocytes, can optionally comprise a “suicide gene” or “safety switch” that enables killing of substantially all of the modified immune cells when desired. For example, the modified T lymphocytes, in certain embodiments, can comprise an HSV thymidine kinase gene (HSV-TK), which causes death of the modified T lymphocytes upon contact with gancyclovir. In another embodiment, the modified T lymphocytes comprise an inducible caspase, e.g., an inducible caspase 9 (icaspase9), e.g., a fusion protein between caspase 9 and human FK506 binding protein allowing for dimerization using a specific small molecule pharmaceutical. See Straathof et al., Blood 1 05 (11): 4247-4254 (2005).

In certain embodiments, an inhibitor of UHRF1 provided herein is administered to a subject with various types or stages of multiple myeloma in combination with chimeric antigen receptor (CAR) T-cells. In certain embodiments the CAR T cell in the combination targets B cell maturation antigen (BCMA), and in more specific embodiments, the CAR T cell is bb2121 or bb21217. In some embodiments, the CAR T cell is JCARH125.

In certain embodiments, an inhibitor of UHRF1 provided herein is administered to a subject with various types or stages of multiple myeloma in combination with a deacetylase inhibitor, such as a histone deacetylase inhibitor (HDAC inhibitor). Suitable DAC or HDAC inhibitors include, for example, 1) hydroxamic acid derivatives; 2) short-chain fatty acids (SCFAs); 3) cyclic tetrapeptides; 4)benzamides; 5) electrophilic ketones; and/or any other class of compounds capable of inhibiting histone deacetylase. In certain embodiments, the HDAC inhibitor is, for example, Suberoylanilide Hydroxamic Acid (SAHA) or LAQ 824. In certain embodiments, the HDAC is panobinostat. In certain embodiments, the HDAC inhibitor is panobinostat, and is used in combination with bortezomib and dexamethasone.

In certain embodiments, an inhibitor of UHRF1 provided herein is administered to a subject with various types or stages of multiple myeloma in combination with an anti-SLAMF7 antibody. In certain embodiments, the anti-SLAMF7 antibody is elotuzumab. In certain embodiments, the anti-SLAMF7 antibody is elotuzumab, and is used in combination with lenalidomide and dexamethasone.

In certain embodiments, an inhibitor of UHRF1 provided herein is administered to a subject with various types or stages of multiple myeloma in combination with an antibody-drug conjugate, i.e., an antibody conjugated to a conjugation moiety or an agent such as a label or toxin. A conjugation moiety can be any conjugation moiety deemed useful to one of skill in the art. For instance, a conjugation moiety can be a polymer, such as polyethylene glycol, that can improve the stability of the antibody in vitro or in vivo. A conjugation moiety can have therapeutic activity, thereby yielding an antibody-drug conjugate. A conjugation moiety can be a molecular payload that is harmful to target cells. A conjugation moiety can be a label useful for detection or diagnosis. In certain aspects, a conjugation moiety is linked to the antibody via a direct covalent bond. In certain aspects, a conjugation moiety is linked to the antibody via a linker. In particular aspects, a conjugation moiety or a linker is attached via one or more non-natural amino acids of an antibody.

In certain embodiments, an inhibitor of UHRF1 provided herein is administered to a subject with various types or stages of multiple myeloma in combination with a nuclear export inhibitor, also known as a selective inhibitor of nuclear export (SINE). In certain embodiments, the nuclear export inhibitor is leptomycin B. In certain embodiments, the nuclear export inhibitor is an exportin 1 (XPO1) inhibitor, such as Selinexor (KPT-330). In certain embodiments, the nuclear export inhibitor is Selinexor and is used in combination with dexamethasone.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

### Example 1. Materials and Methods

shRNA Knockdown

Doxycycline (Dox)-inducible shRNA constructs were generated by Cellecta (Mountain View, CA, USA) using pRSITEP-U6Tet-(sh)-EF1-TetRep-2A-Puro plasmid. Briefly, 293 FT cells were co-transfected with lentiviral packaging plasmid mix (Cellecta, Cat #CPCP-K2A) and pRSITEP-shRNA constructs. Viral particle was collected 48 after transfection and then concentrated 10-fold by Amicon Ultra-15 centrifugal filters. For infections, cells were incubated overnight with concentrated viral supernatants in the presence of 8 μg/mL polybrene. Cells were then washed to remove polybrene. At 48 hours post-infection, cells were selected with puromycin (2 μg/mL) for more than 3 weeks before experiments. H929 and MM1.S cells were induced with Dox at concentration of 20 and 10 ng/mL, respectively, with indicated periods of times. The shRNA target sequences were:

shLuciferase (Luc):
(SEQ ID NO: 1)
CGCTGAGTACTTCGAAATGTC;
shNT:
(SEQ ID NO: 2)
CAACAAGATGAAGAGCACCAA;
shIKZF1-1094:
(SEQ ID NO: 3)
CGTGATGGACCAAGCCATCAA;
shIKZF1-2908:
(SEQ ID NO: 4)
CCCAGCATCTCTGTTGCTAAC;
shIKZF3-3:
(SEQ ID NO: 5)
GCCTCTCTCCCAACAATAGTG;
shIKZF3-6:
(SEQ ID NO: 6)
GTAACCTCCTCCGCCACATTA;
shZFP91-76:
(SEQ ID NO: 7)
CGCGACTCCTATGCATAGAAA;
shZFP91-77:
(SEQ ID NO: 8)
GCAGACTCCTTCTACCAGTTT;
shCRBN-60:
(SEQ ID NO: 9)
CAGGATAGTAAAGAAGCCAAA;
shUHRF1-4:
(SEQ ID NO: 10)
GTGCCAGCACAACGTGTGCAA;
shUHRF1-15:
(SEQ ID NO: 11)
ATGTGGGATGAGACGGAATTG.

### RNA Extraction, Reverse Transcription and Real-Time PCR

Total RNA was extracted by RNeasy Mini Kit (Qiagen) and reverse transcribed by iScrip cDNA Synthesis Kit (Bio-rad). Quantitative real-time PCR (qPCR) was conducted on a VillA 7 System using the Power SYBR Green PCR Master Mix (Applied Biosystems). Gene expression values were calculated by normalization to Actb using the comparative CT method.

### ChIP-qPCR

ChIP-qPCR was performed using ChIP-IT Express Enzymatic kit (Active Motif) according to the manufacture manual with some modifications. Briefly, cells were cross-linked with 1% formaldehyde for 10 minutes, and cross-linking was stopped with 125 mM glycine for 5 minutes. After PBS washed, cells were resuspended in cell lysis buffer at concentration of 2×10⁷ cells/mL and incubated on ice for 30 min. Nuclei were then pelleted and resuspended in 350 μL of digestion buffer and then subject to sonication for 5 rounds (for each round: 1 sec on/1 sec off, 10 sec, 30% output). Chromatin was then digested with diluted enzymatic shearing cocktail (200 U/mL) for at 37° C. for 25 minutes. Chromatin containing twenty microgram of DNA was subjected to immunoprecipitation overnight at 4° C. with 5 μL of either of the following antibodies: anti-Aiolos (Cell Signaling Technology, CST, #15103), anti-H3K27ac (CST, #8173), anti-histone H3 (CST, #4499) or normal rabbit IgG (CST, #2729). The immunoprecipitates were then washed and eluted according to the manual. DNA was purified using a PCR purification kit (Qiagen) and analyzed by qPCR using the Power SYBR Green PCR Master Mix.

Procedures afterward were performed according to the manufacture manual. Eluted DNA was purified using a PCR purification kit (Qiagen) and analyzed by quantitative real-time PCR on the Viia 7 Real Time PCR System using the Power SYBR Green PCR master mix (Applied Biosystems). Statistical differences were calculated using a two-way unpaired Student's t-test.

### (3H)-Thymidine Incorporation Assay

(3H)-thymidine incorporation assay was performed as previously described (PMID: 23252516) with minor modifications. Briefly, 1×10⁴ or 2×10⁵ multiple myeloma (MM) cells were cultured in 96-well culture plates in complete media with the indicated concentration of pomalidomide (POM) or DMSO control for 3 days. 37 kBg (3H)-thymidine was added to each will at the final 6 hour of incubation.

### 5-mc Dot Blot

Genomic DNA of cells were isolated by QIAamp DNA Mini Kit (Qiagen) and treated with RNase A. Extracted genomic DNA was heated at 95° C. for 10 minutes in the presence of 0.4 N NaOH and 10 mM EDTA and then neutralized with equal volume of 2 M ammonium acetate. Serial diluted genomic DNA was spotted on to nitrocellulose membrane (pre-soaked with 6X saline-sodium citrate (SSC) buffer) using Bio-Dot® SF Microfiltration Apparatus (Bio-rad) according to the manufacture manual. Genomic DNA was then UV crosslinked to nitrocellulose membrane. After blocking, membrane was incubated with anti-5mC antibody (1 μg/mL, Millipore MABE146) at 4° C. overnight, followed by incubation with IRDye® 680RD goat anti-Mouse IgG (H+L) secondary antibody (1:10,000; LI-COR).

### Western Blotting

Cells were lysed with cell lysis buffer (50 mM TrisHCl pH7.4, 250 mM NaCl, 0.5% Triton X100, 10% glycerol) supplemented with protease inhibitor cocktail. Cell lysates were subjected to sonication to breakdown nuclei and reduce viscosity caused by released genomic DNA. The protein concentration was measured by a Bradford Protein Assay (Bio-Rad). Samples were mixed with SDS sample buffer at 95° C. for 5 minutes. Whole cell lysates were resolved on NuPAGE™ 4-12% Bis-Tris Midi Protein Gels (Invitrogen) and transferred onto nitrocellulose membranes, which were then subjected to blocking in Intercept (TBS) Blocking Buffer (LI-COR). Proteins of interested were detected incubation with primary antibodies listed below at 4° C. overnight. Then the membrane was incubated with either IRDye 680RD goat anti-Mouse IgG (H+L) or IRDye 800CW goat anti-Rabbit IgG (H+L) (1:10,000; LI-COR). Bends were visualized by Odyssey Imaging System (LI-COR).

### Example 2. Correlation of Down Regulation of UHRF1 Gene Expression with Sensitivity of Multiple Myeloma to Pomalidomide

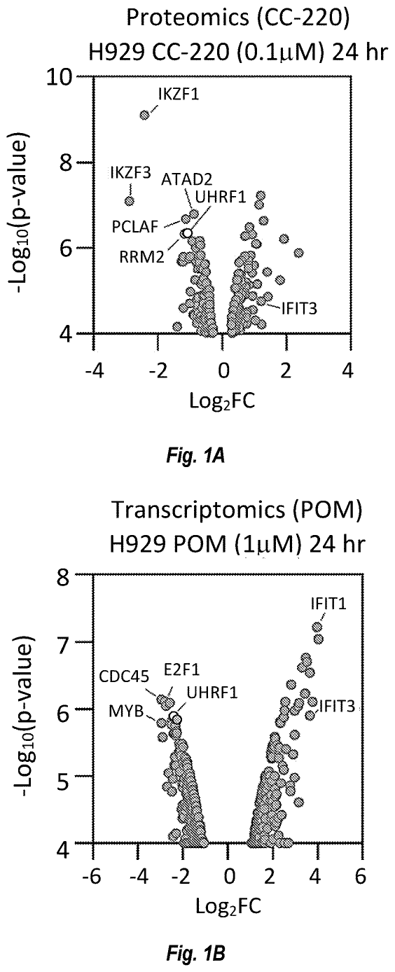

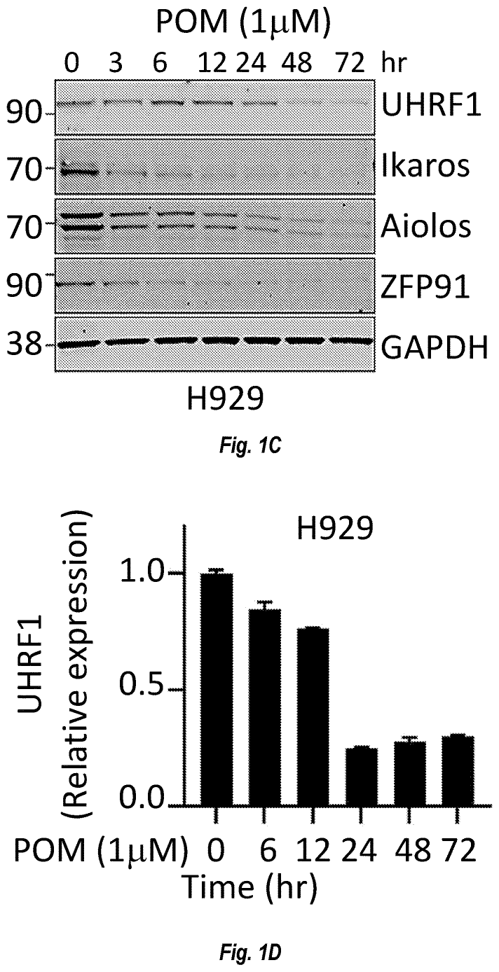

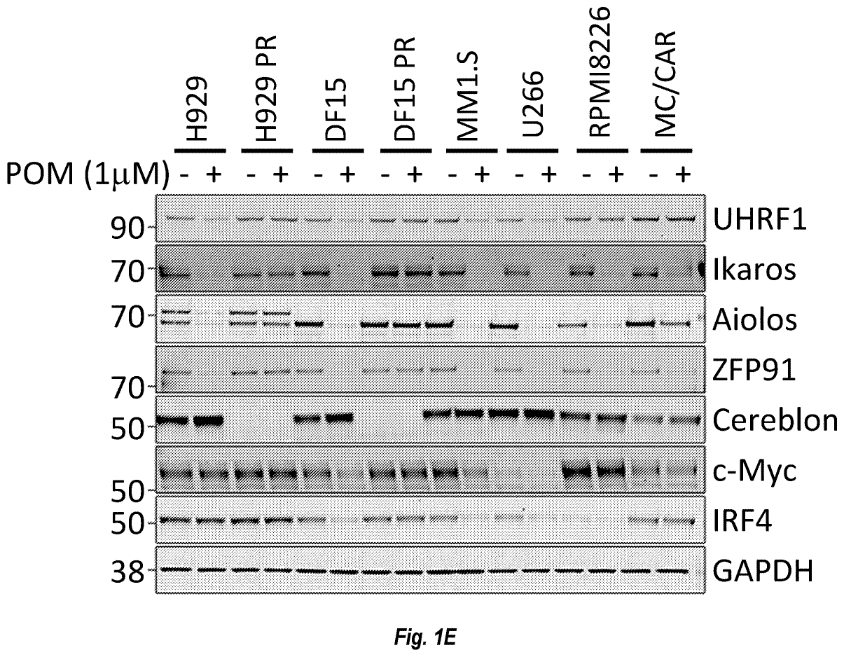

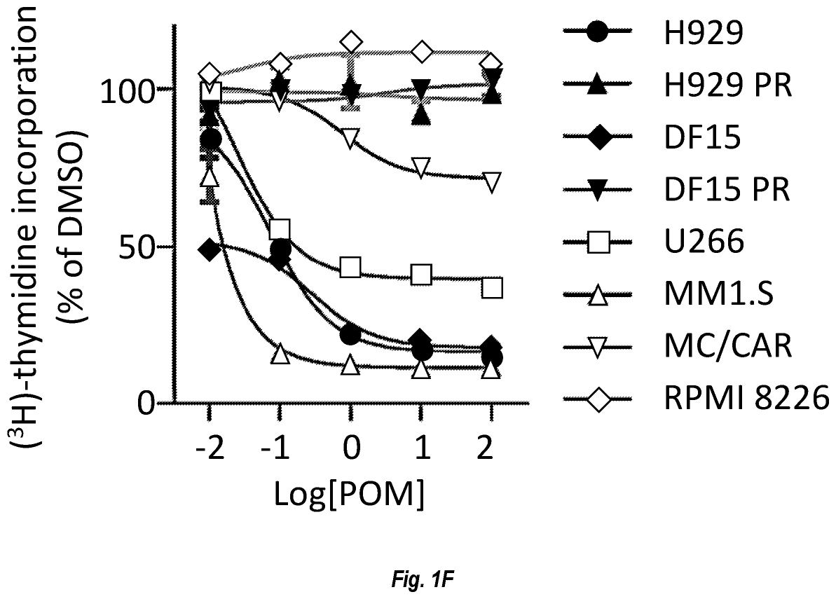

To identify novel effectors that mediate the effects of immunomodulatory drugs that bind to cereblon (IMiD), a proteomic database was searched for proteins that are downregulated by CC-220, a cereblon E3 ligase modulator with higher potency than pomalidomide (POM), in drug sensitive H929 cells. In addition to the well-known celebron substrates, Ikaros (IKZF1) and Aiolos (IKZF3), UHRF1 was identified as one of the most significantly downregulated proteins, as shown in FIG. 1A. In a separate study, where transcriptome of POM (1 μM, 24 hour)-treated H929 cells were profiled by affymetrix microarray, drastic induction of interferon response genes, IFIT1 and IFIT3 was observed, which were shown to be suppressed by Ikaros and Aiolos (Also see FIG. 1B). Interestingly, UHRF1 was found to be one of the most significantly downregulated genes in addition to several cell cycle regulators such as E2F1, MYB and CDC45. Given that UHRF1 has been reported to be oncogenic in other cancer types, including MM (Gu et al., 2020, BMC Biol, 18:33), the functions of UHRF1 in MM and the mechanisms underlying its gene regulation in response to immunomodulator drugs that binds to cereblon was explored.

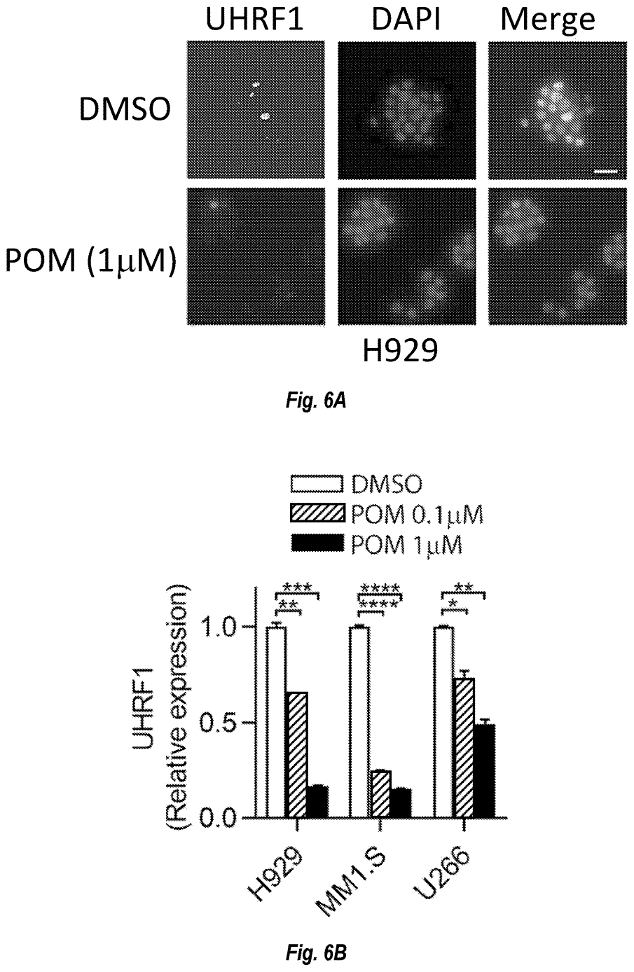

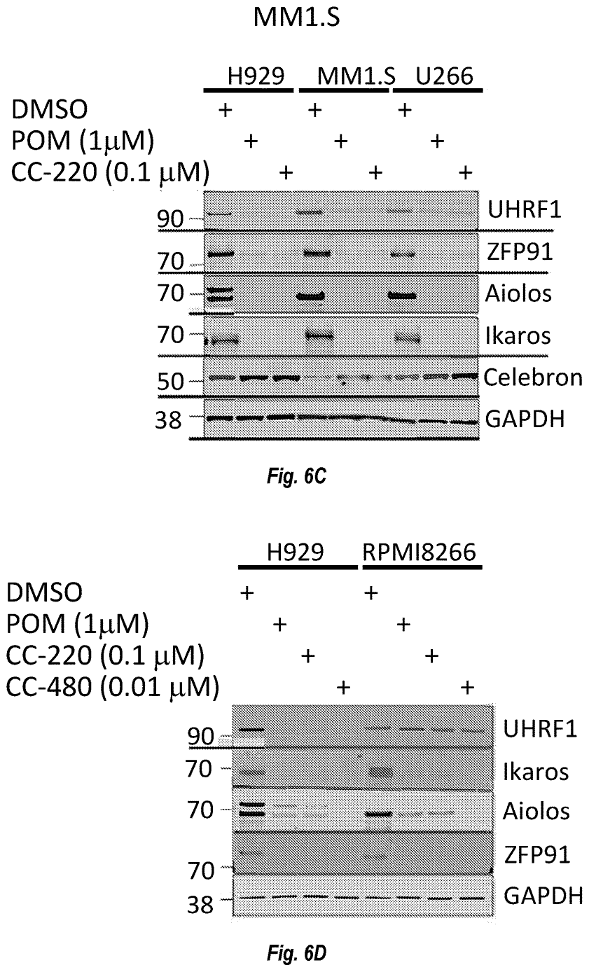

These results show that UHRF1 is downregulated by POM at the transcription level, suggesting it is not a celebron substrate, which is expected to be downregulated at the protein but not mRNA level in response to immunomodulatory drugs, such as POM. To delineate the temporal dynamics of UHRF1 expression and cereblon substrates degradation, a time course study on H929 cells treated with POM was performed. As shown in FIGS. 1C and 1D, while Ikaros, Aiolos and ZFP91 proteins were degraded as early as 3 hours following POM (1 μM) treatment, it took up to 24 and 48 hours for profound reduction of UHRF1 mRNA and protein levels, respectively. The observation that degradation of the cereblon substrates preceded downregulation of UHRF1 transcripts and UHRF1 protein, supported the hypothesis that UHRF1 protein is likely not a substrate of cereblon complex, rather a transcriptional target down-regulated by POM-elicited events. In line with the notion, immunocytochemistry assay showed that while UHRF1 protein is predominantly localized in the nucleus of H929 cells, it was nearly undetectable 48 hours after POM treatment (see FIG. 6A). A dose dependent downregulation of UHRF1 mRNA in several POM sensitive cell lines (H929, MM1.S and U266) treated with POM was also observed (see FIG. 6B). Moreover, administration of CC-220 (0.1 μM) led to similar downregulation of UHRF1 protein in these cells at 72 hours (see FIG. 6C). These results suggest downregulation of UHRF1 transcript is a common response when drug sensitive MM cells are exposed to immunomodulatory drugs that bind to cereblon.

Tests to determine UHRF1 and other substrate expression levels in response to POM in various MM cells with different drug sensitivities showed that at 72 hours post POM treatment, UHRF1 and the substrates, Ikaros, Aiolos and ZFP91, were downregulated in sensitive cell lines (H929, MM1.S, DF15 and U266), but not the cell lines (H929 PR and DF15 PR) that acquired resistance through diminished cereblon expression (see FIGS. 1E and 1F). Expression of IRF4 and c-Myc, whose downregulation have been linked to antimyeloma activity of immunomodulatory drugs that bind to cereblon (Lopez-Girona et al., 2012, Leukemia 26:2326; Zhang et al., 2013, Br J Haematol, 160:487) was then examined. Interestingly, in the experimental condition, POM treatment downregulated IRF4 and c-Myc in MM1.S, DF15 and U266, but not H929 cells, although all these lines are drug sensitive. These results suggest the anti-myeloma activities of immunomodulatory drugs that bind to cereblon are not solely dependent on the downregulation of IRF4 and c-Myc, which is consistent the notion of a previous studies [5,6]. Strikingly, POM degraded substrates without affecting UHRF1 protein levels in MC/CAR and RPMI 8226 cells which are less sensitive to or intrinsically resistant to the drug, respectively. Accordingly, POM-mediated degradation of cereblon substrates correlates with downregulation of UHRF1 in drug sensitive but not resistant MM cell lines. Taken together, these results not only suggest that compared to substrates degradation, UHRF1 downregulation appeared to be as a more reliable marker for drug sensitivity, but also indicate that diminished UHRF1 expression contributes to drug-mediated myeloma inhibition.

### Example 3. Positive Regulation of UHRF1 Expression by Ikaros and Aiolos in Drug Sensitive MM Cells

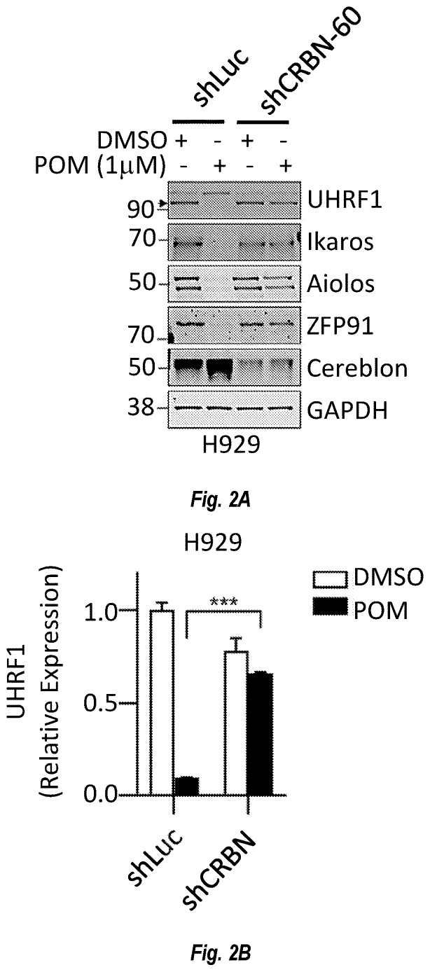

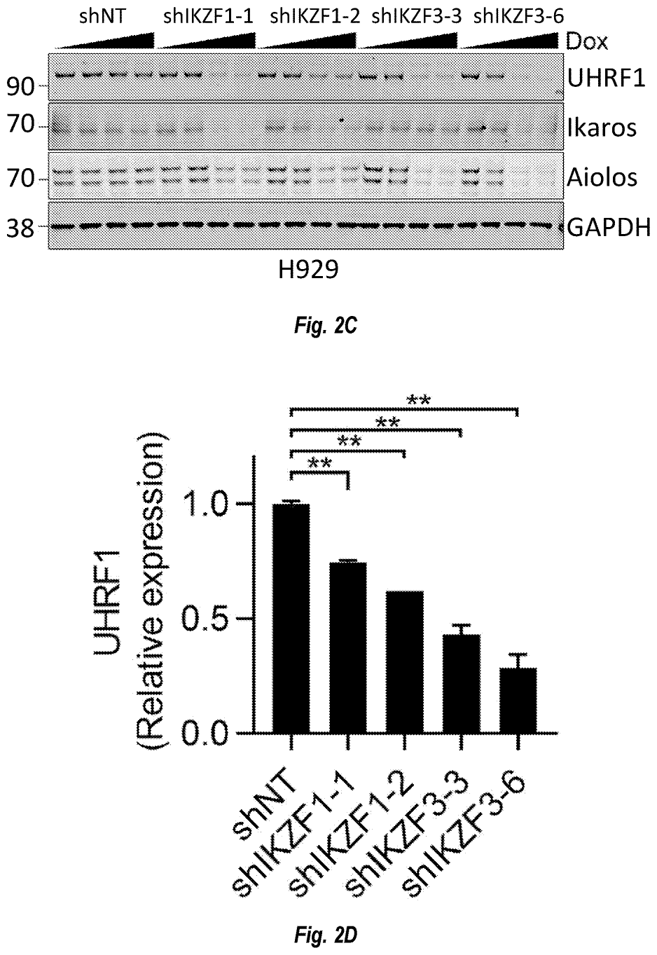

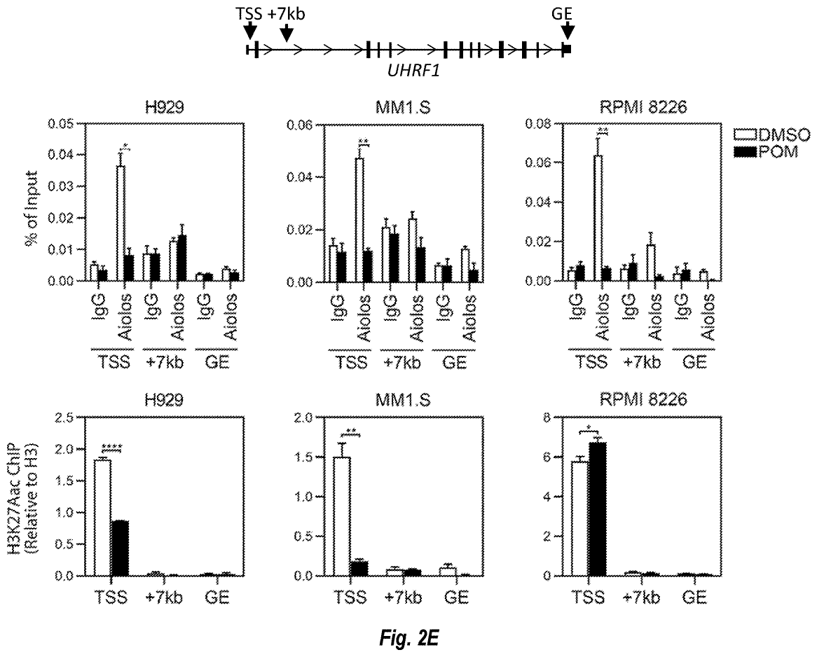

The observation that UHRF1 was downregulated by POM in sensitive cell lines (H929 and DF15) but not in their acquired resistant derivatives (H929 PR and DF15 PR) with reduced cereblon expression (FIG. 1E) suggested that downregulation of UHRF1 is cereblon dependent. To test the hypothesis, the expression of UHRF1 in H929 cells with or without cereblon knockdown was determined. As shown in FIG. 2A, knockdown of cereblon rescued the degradation of the substrates and the downregulation of UHRF1 protein by POM. The rescue was at UHRF1 mRNA level, in line with previous observations that POM downregulates UHRF1 transcripts (FIG. 2B).

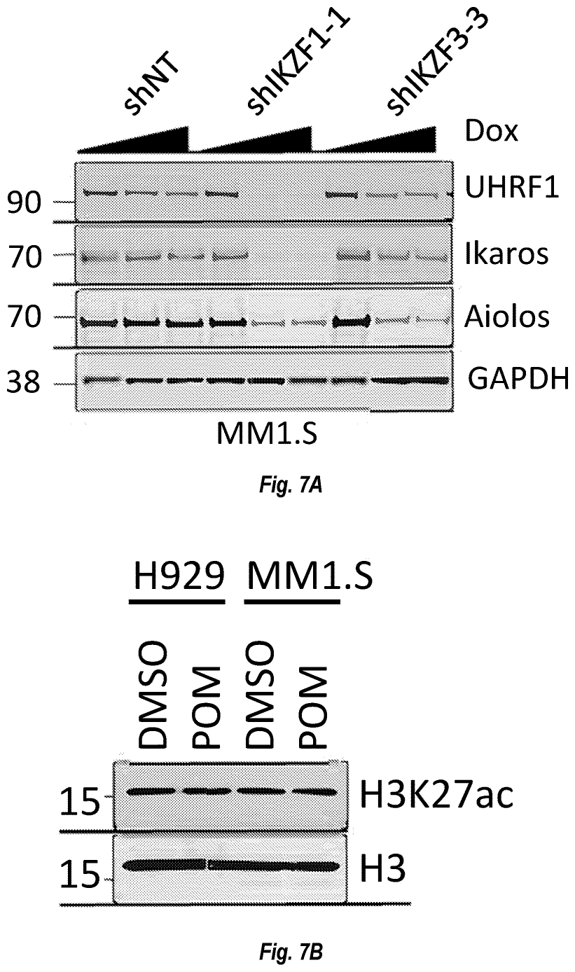

To determine which substrates were responsible for UHRF1 expression, H929 stable cells with doxycycline (Dox)-inducible shRNA targeting Ikaros or Aiolos was generated. Knockdown of either of the two substrates caused reduction of UHRF1 protein (FIG. 2C) and mRNA (FIG. 2D) in H929 cells. Consistent with previous reports (Lu et al, 2014, Science, 343:305; Fedele et al., 2018, Blood, 132:2166; Bjorklund et al., 2015, Blood Cancer J, 5: e354), depletion of either Ikaros or Aiolos led to downregulation of the other (FIG. 2C). Further tests were done to examine whether UHRF1 expression is similarly dependent on Ikaros and Aiolos in MM1.S cells, another IMiDs-sensitive cell lines. Data revealed drastic reduction of UHRF1 in Ikaros knockdown MM1.S cells. Knockdown of Aiolos led to relatively mild reduction of UHRF1 as compared to non-targeting shRNA (shNT) control, which was likely due to a moderate Aiolos knockdown efficiency in this cell line (see FIG. 7A). Taken together, these results suggest UHRF1 transcripts are positively regulated by Ikaros and Aiolos in myeloma cells that are sensitive to cereblon modulators.

The dependency of UHRF1 gene expression on Ikaros and Aiolos is reminiscent of IRF4 gene, whose expression is upregulated by Ikaros through directly binding to its promoter in MM cells [5]. To test whether UHRF1 gene expression is regulated through similar mechanisms, chromatin immunoprecipitation (ChIP)-qPCR experiments were performed to determine if Aiolos occupies the UHRF1 promoter. As shown in FIG. 2E, compared to control IgG, an antibody against Aiolos significantly pulled down more promoter or transcription start site (TSS) DNA, but not DNA of the gene body (+7 kb) or gene end (GE) of UHRF1. Importantly, the UHRF1 promoter occupancies of Aiolos were drastically reduced in POM-treated cells, confirming the specificity of the ChIP assay. Similar results were observed in MM1.S cells. Surprisingly, Aiolos also bound to the promoter of UHRF1 in RPMI 8226 cells, in which UHRF1 is not regulated by Aiolos (see FIG. 1E). Studies were conducted to attempt to validate if Ikaros similarly binds to the UHRF1 promoter; however, a satisfactory signal-to-noise ratio by ChIP-qPCR using commercially available antibodies was not obtained. To further delineate how POM regulates UHRF1 gene expression, H3K27ac marks at the UHRF1 promoter under POM treatment were examined. Results demonstrated that POM treatment drastically reduced the H3K27ac level at the UHRF1 TSS of H929 and MM1.S, but not RPMI 8226 cells (see FIG. 2E). These results suggest POM treatment reduced UHRF1 gene expression through reduced H3K27ac marks in drug sensitive but not resistant cells. The global levels of H3 and H3K27ac were not affected by POM (see FIG. 7B), suggesting that the decreased H3K27ac mark at UHRF1 promoter was not due to global reduction of H3K27ac. Taken together, the data revealed that Aiolos bound to the UHRF1 promoter and stimulated its gene expression through maintaining the H3K27ac activation mark in drug sensitive cells.

### Example 4. Function of UHRF1 in Both Drug Sensitive and Resistant MM Cell Lines

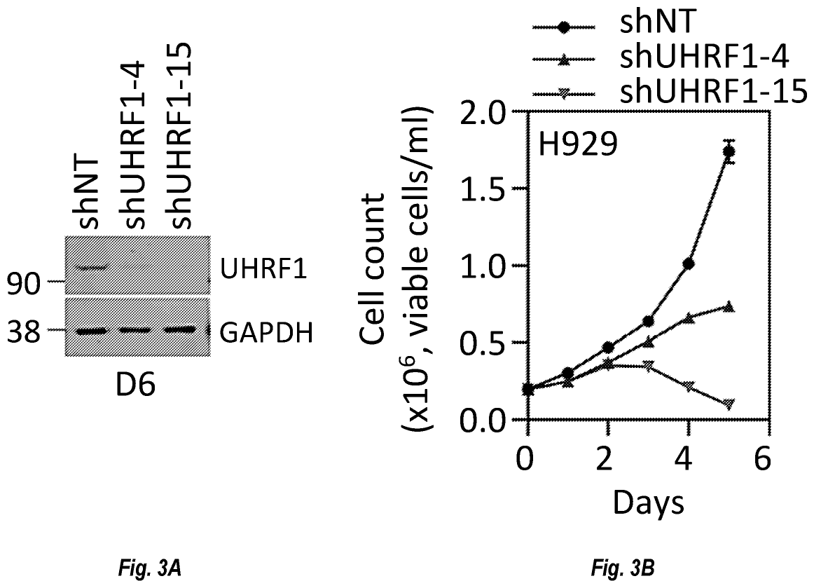

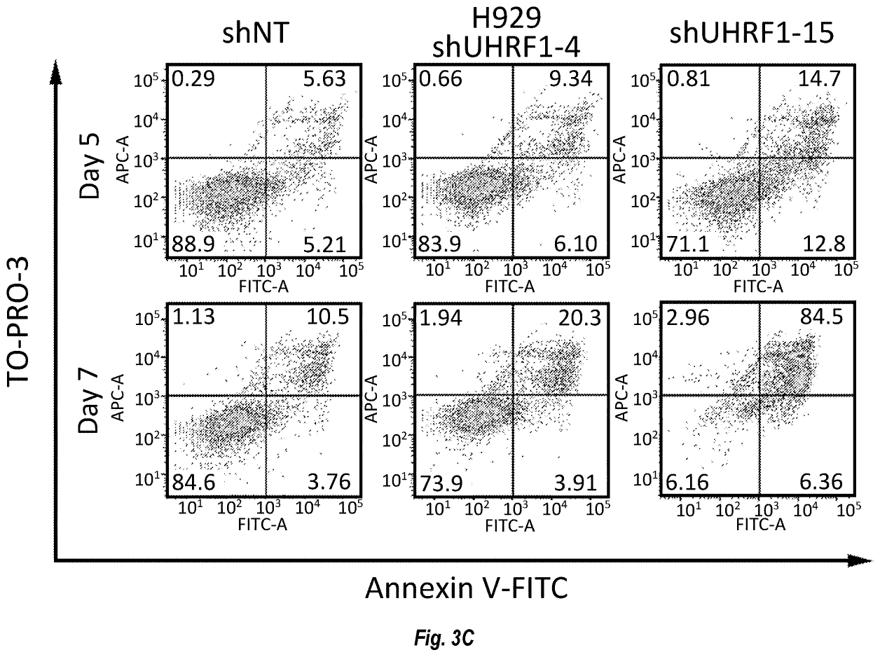

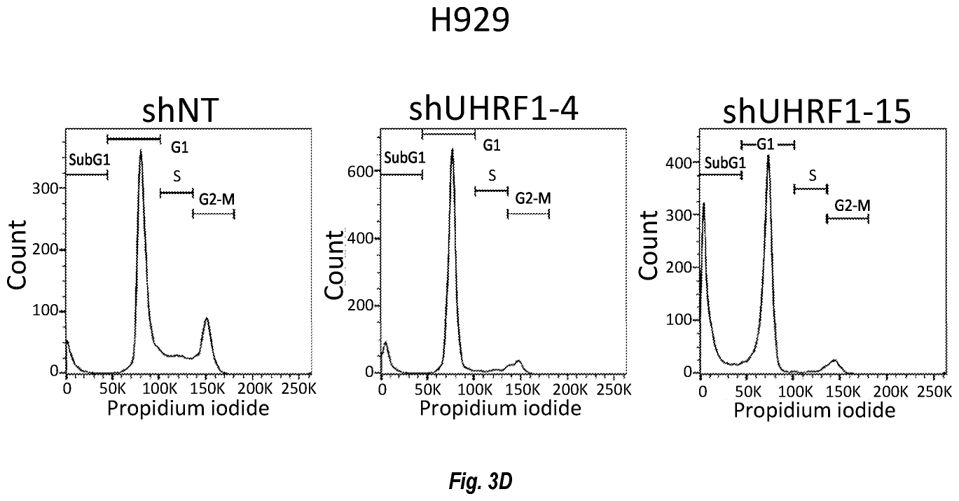

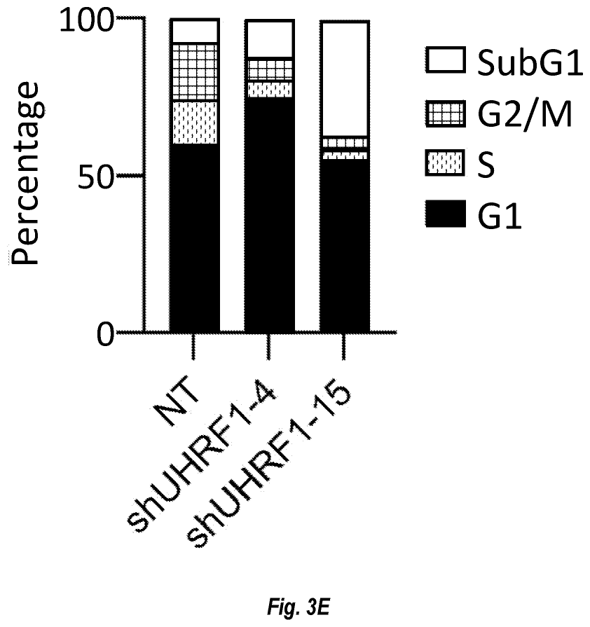

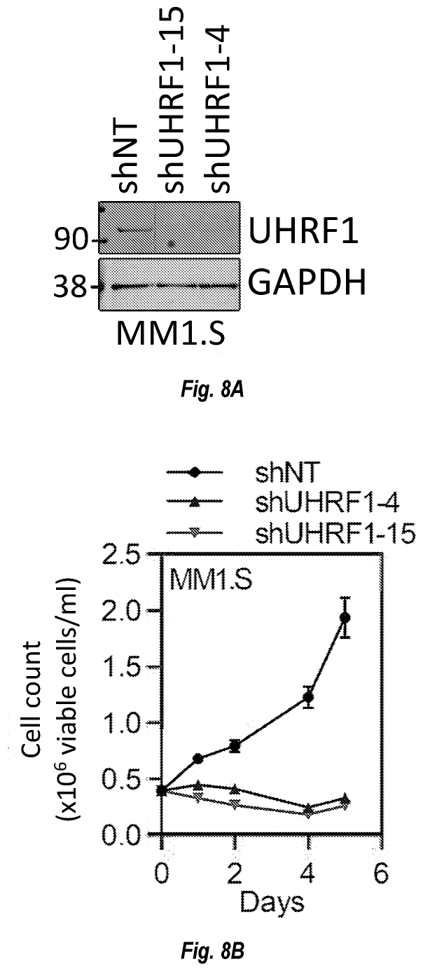

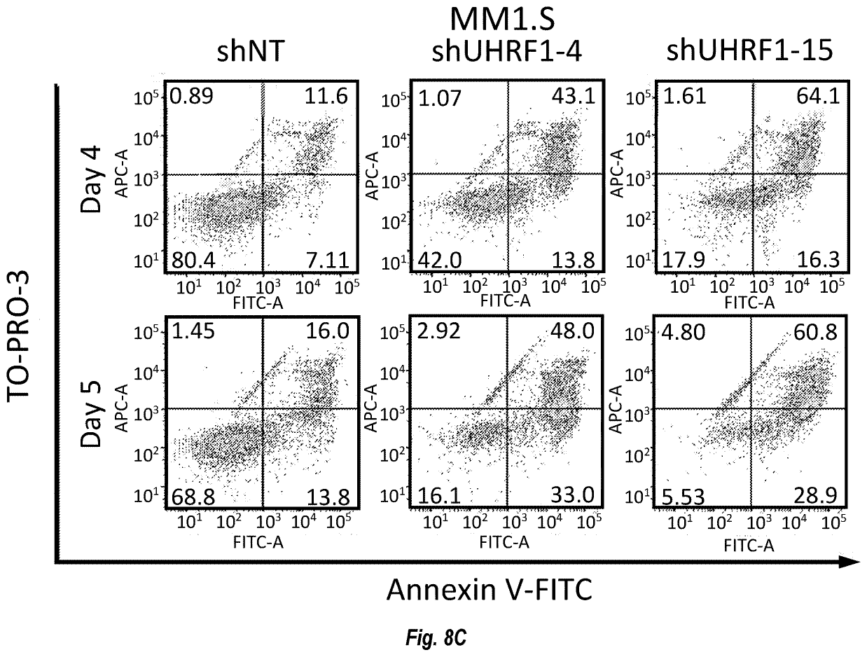

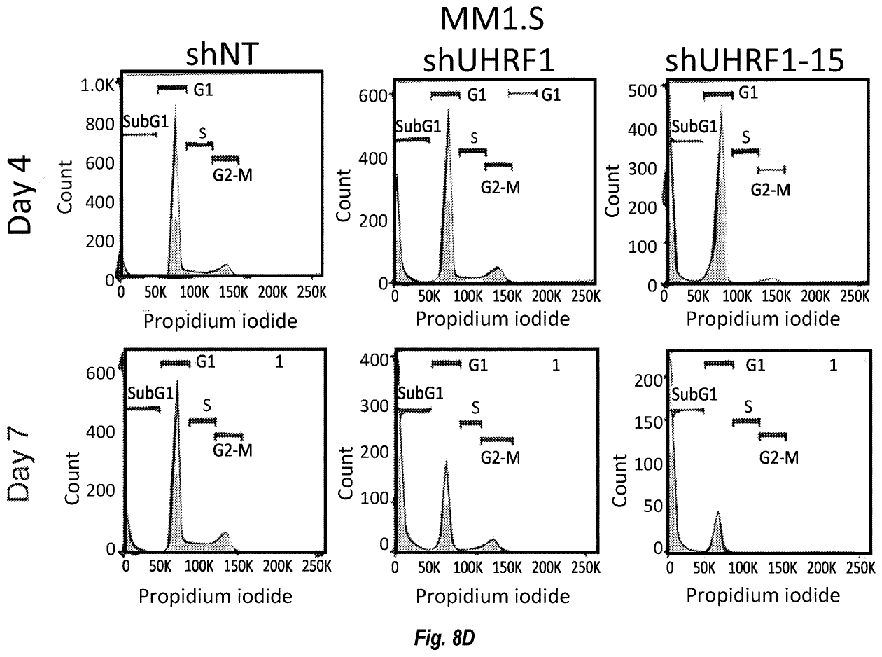

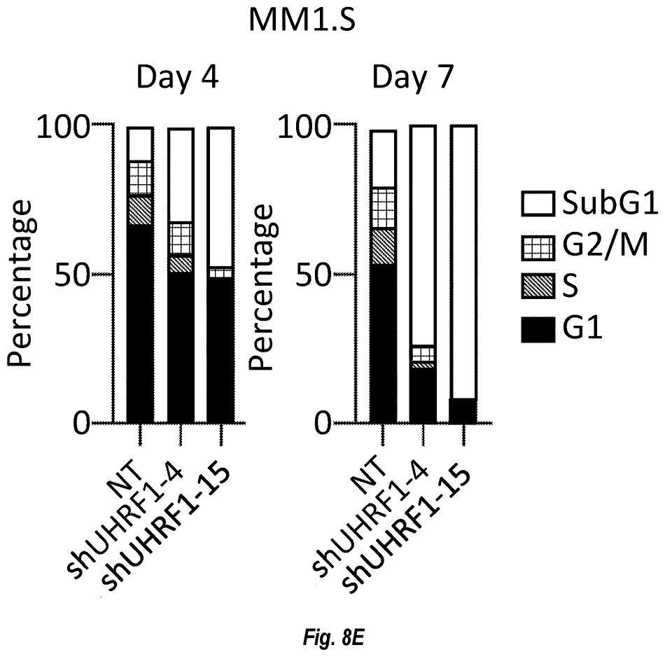

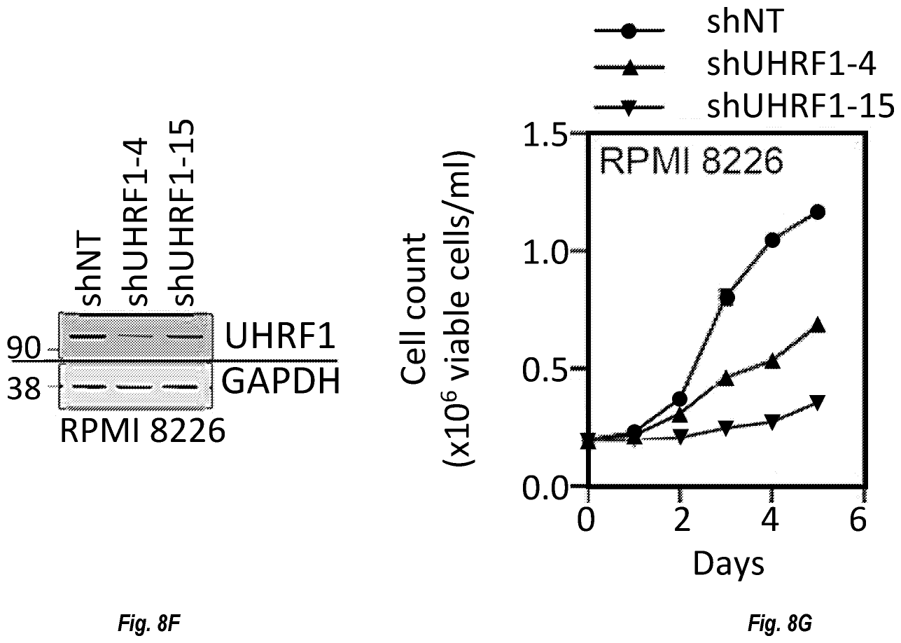

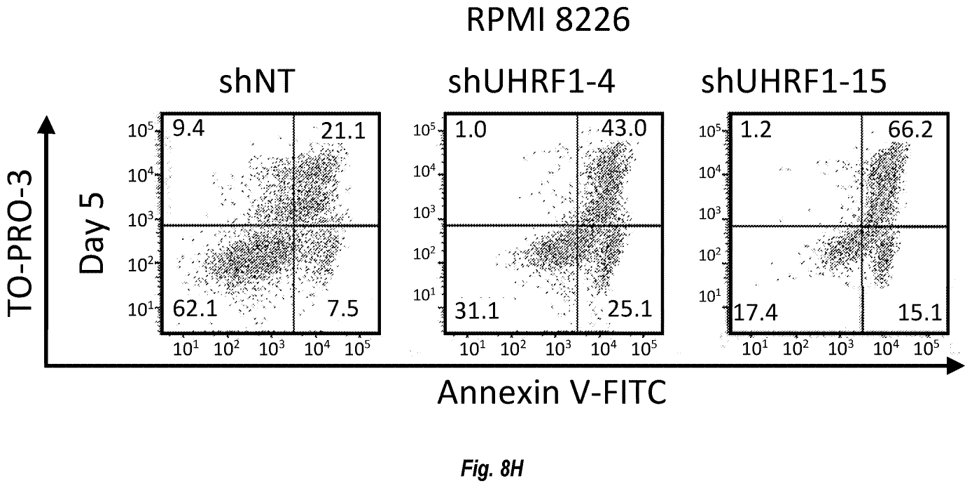

The observation that POM selectively downregulated UHRF1 in sensitive but not resistant MM, prompted us to characterize the roles of UHRF1 in MM. To this end, stable H929 cells in which the UHRF1 can be inducibly knocked down by doxycycline were generated. As shown in FIG. 3A, although both shUHRF1-4 and shUHRF1-15 efficiently downregulated UHRF1, the effect of the latter shRNA was more pronounced. A cell proliferation assay was performed by inducing non-target or UHRF1 shRNA expression for 3 days, followed by seeding and counting cells over the course of 5 days. Data showed that UHRF1 knockdown led to reduced cell number and, importantly, the extent of reduction correlated with the knockdown efficiency (see FIG. 3B). Experiments were then conducted to confirm that the reduced cell number was a consequence of cell death and/or cell cycle arrest. Annexin V-FITC/TO-PRO-3 staining showed that UHRF1 knockdown for 7 days increased apoptotic (Annexin V positive) cells by ~10% and ~70% in cells expressing shUHRF1-4 and shUHRF1-15, respectively (see FIG. 3C). On the other hand, cell cycle analysis showed that shUHRF1-4 expression resulted in cell cycle arrest, with increased G1 and reduced G2/M populations. Consistent with the observation of apoptosis assay, induction of shUHRF1-15 drastically increased sub-G1 population (see FIGS. 3D and 3E). These results suggested that in H929 cells, modest reduction of UHRF1 mainly caused cell cycle arrest, while more complete depletion of UHRF1 led to massive apoptotic cell death. Similar assays were performed in MM1.S cells and found this cell line was even more sensitive to UHRF1 depletion, evidenced by flattened growth curves, and drastically increased apoptotic and subG1 cell populations in cells treated with either of the two shRNAs (see FIGS. 8A-8E). Further experiments were done to test if UHRF1 is also essential for MM cells that are intrinsically drug resistant. UHRF1 knockdown inhibited proliferation and induced apoptosis in RPMI 8226 cells (see FIGS. 8F-8H). In summary, UHRF1 is essential for proliferation of both drug sensitive and drug resistant MM cell lines.

### Example 5. Effect of UHRF1 Depletion on Interferon Response Genes

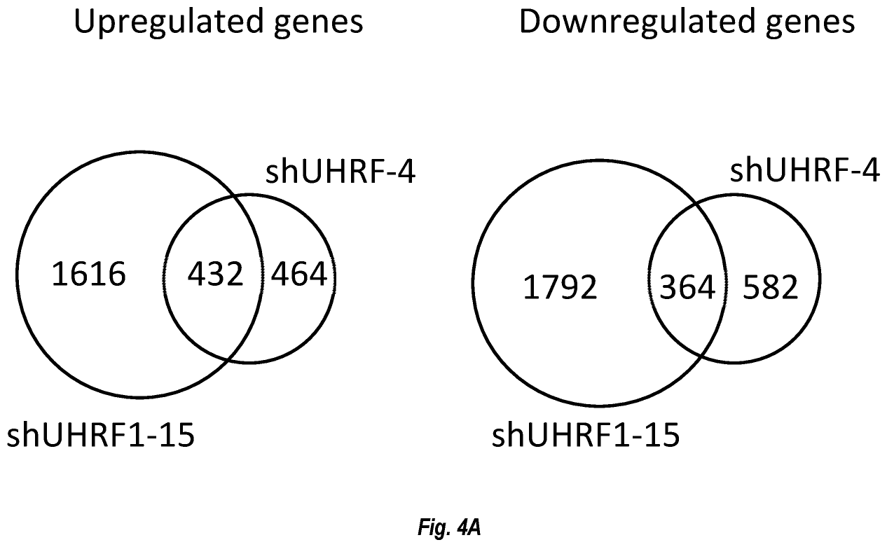

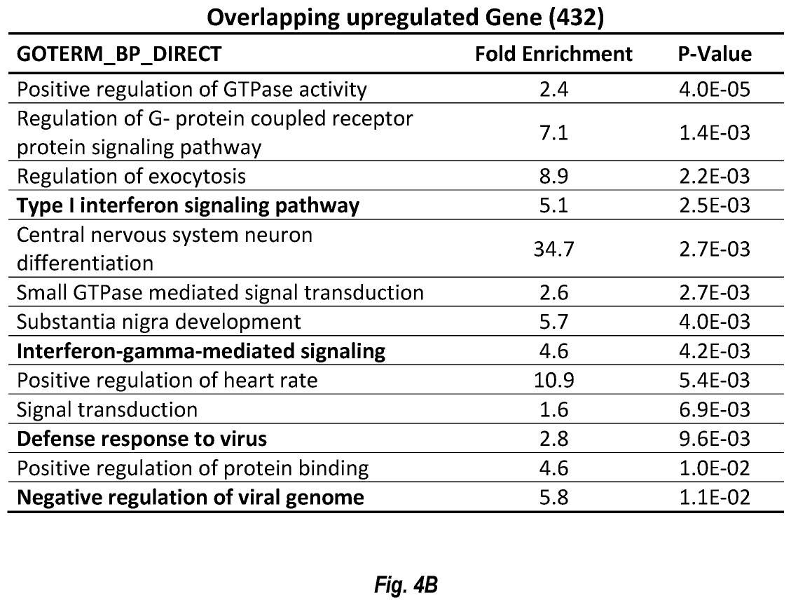

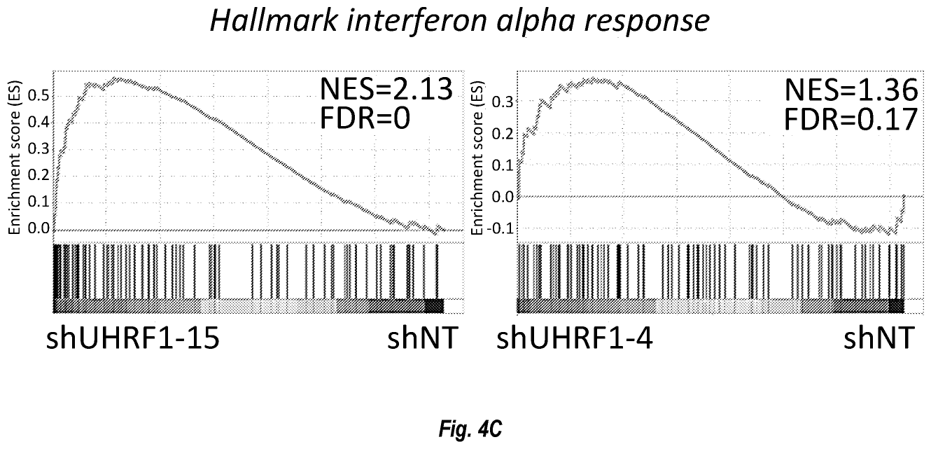

To gain more insight into why UHRF1 is essential for MM cell proliferation, a profile of the transcriptome of UHRF1 knockdown (shUHRF1-4 and shUHRF1-15) and mock knockdown (shNT) in H929 cells with RNA sequencing was done. Results identified a total of 432 genes [log 2FC >0.5, FDR <0.05) commonly upregulated and 364 [log 2FC <−0.5, FDR <0.05] genes commonly downregulated in cells treated with either UHRF1 shRNA (see FIG. 4A). Gene ontology analysis with PANTHER revealed that the 432 upregulated genes were enriched in various biological processes, including interferon signaling pathways and viral defense responses. (See FIG. 4B).

### Example 6. Association of High UHRF1 Expression with Poor Prognosis of MM

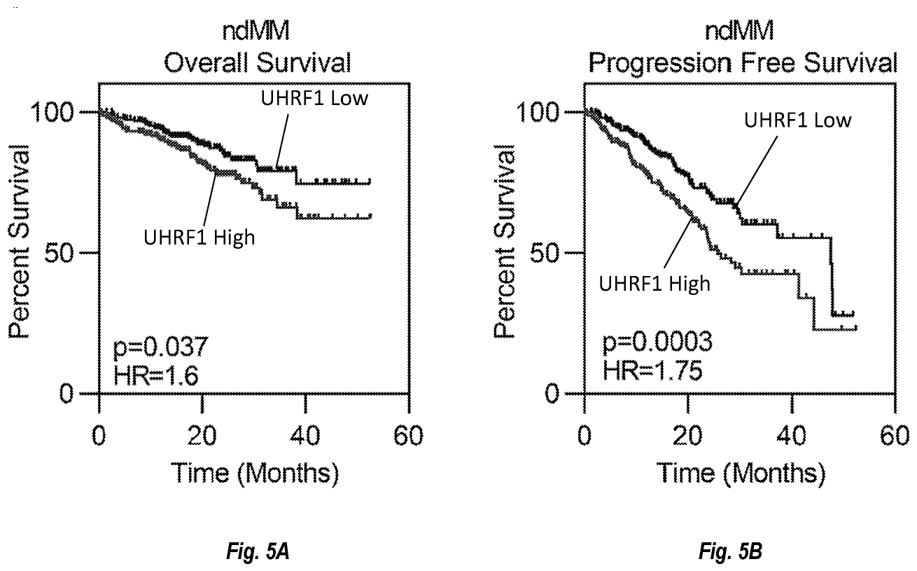

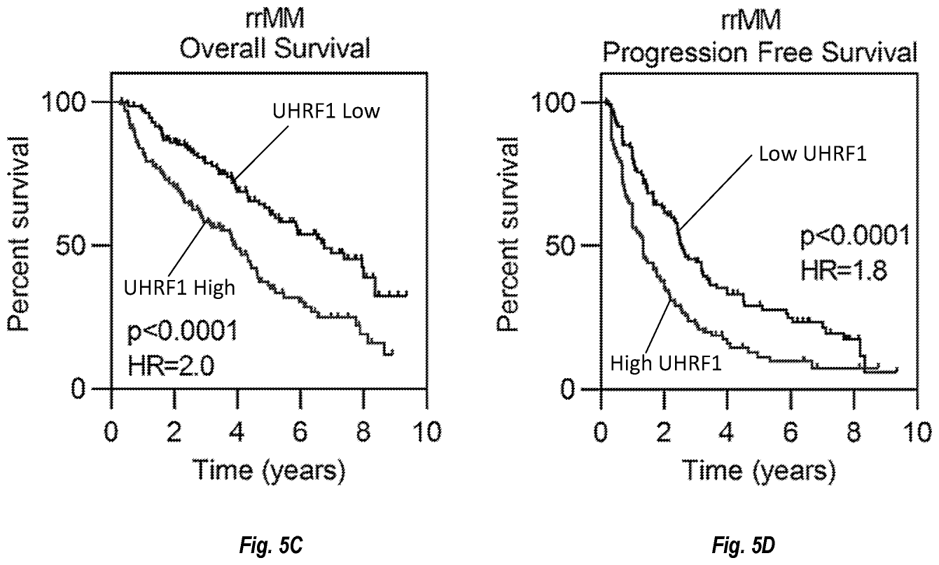

In order to evaluate whether the expression of UHRF1 holds prognosis value in MM patients, data from 514 newly diagnosed MM (ndMM) patients from Myeloma Genome Project (MGP) (Walker, et al., 2019, Leukemia, 33:159) were segmented into UHRF1 high and low expression categories, respectively. Patients with high UHRF1 expression had significantly shorter overall survival (OS) and progression-free survival (PFS; median, 25.7 vs 47.5 months) compared with those with low UHRF1 expression (see FIGS. 5A and 5B). To test whether UHRF1 expression is also prognostic in relapsed/refractory MM (rrMM) patients, the MM-010 dataset, a clinical trial (NCT01712789) for safety of POM in combination with low dose dexamethasone in rrMM patients was examined (Dimopoulos, et al., 2016, Blood, 128:497). Again, results showed patients with high UHRF1 expression had significantly shorter OS (median, 3.87 vs 6.75 years) and PFS (median, 1.35 vs 2.57 years) compared with those with low UHRF1 expression (see FIGS. 5C and 5D). Therefore, high UHRF1 was associated with inferior prognosis in both ndMM and rrMM patients.

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

What is claimed is:

1. A method of treating multiple myeloma, comprising administering to a subject with multiple myeloma a therapeutically effective amount of an inhibitor of Ubiquitin-Like with PHD and Ring Finger Domains 1 (UHRF1), wherein the multiple myeloma is resistant to at least one therapeutic agent.

2. The method of claim 1, wherein the multiple myeloma is resistant to at least one IMiDR.

3. The method of claim 2, wherein the multiple myeloma is resistant to at least one IMiD® selected from lenalidomide, thalidomide, pomalidomide, avadomide, and iberdomide.

4. A method of treating multiple myeloma, comprising administering to a subject with multiple myeloma a therapeutically effective amount of an inhibitor of Ubiquitin-Like with PHD and Ring Finger Domains 1 (UHRF1) and at least one additional therapeutic agent.

5. The method of claim 3, wherein the multiple myeloma is resistant to at least one IMiD®.

6. The method of claim 5, wherein the multiple myeloma is resistant to at least one IMiD® selected from lenalidomide, thalidomide, pomalidomide, avadomide, and iberdomide.

7. The method of any one of claims 4-6, wherein at least one additional therapeutic agent is selected from a chemotherapy agent, a steroid, an immunomodulating agent, a proteasome inhibitor, a histone deacetylase inhibitor, an anti-CD38 antibody, an anti-SLAMF7 antibody, an antibody-drug conjugate, a nuclear export inhibitor, a BCMA-directed T-cell engager, an NK cell engager, and a CAR-T therapy.

8. The method of any one of claims 4-7, wherein at least one additional therapeutic agent is selected from lenalidomide, thalidomide, pomalidomide, avadomide, iberdomide, bortezomib, carfilzomib, ixazomib, panobinostat, melphalan, vincristine, cyclophosphamide, etoposide, doxorubicin, and bendamustine, dexamethasone, prednisone, daratumumab, isatuximab, elotuzumab, belantamab mafodotin-blmf, Selinexor, pamidronate, zoledronic acid, and denosumab.

9. The method of any one of claims 4-8, wherein the at least one additional therapeutic agent is selected from:
a) lenalidomide;
b) iberdomide;
c) (S)-4-(4-(4-(((2-(2,6-dioxopiperidin-3-yl)-1-oxoisoindolin-4-yl)oxy)methyl)benzyl) piperazin-1-yl)-3-fluorobenzonitrile;
d) (i) lenalidomide, pomalidomide, or thalidomide; and (ii) dexamethasone;
e) (i) carfilzomib, ixazomib, or bortezomib; (ii) lenalidomide; and (iii) dexamethasone;
f) (i) bortezomib or carfilzomib; (ii) cyclophosphamide; and (iii) dexamethasone;
g) (i) elotuzumab or daratumumab; (ii) lenalidomide; and (iii) dexamethasone;
h) bortezomib, liposomal doxorubicin, and dexamethasone;
i) panobinostat, bortezomib, and dexamethasone;
j) elotuzumab, bortezomib, and dexamethasone;
k) melphalan and prednisone, with or without thalidomide or bortezomib;
l) vincristine, doxorubicin, and dexamethasone;
m) dexamethasone, cyclophosphamide, etoposide, and cisplatin; and
n) dexamethasone, thalidomide, cisplatin, doxorubicin, cyclophosphamide, and etoposide, with or without bortezomib.

10. The method of any one of the preceding claims, wherein the inhibitor of UHRF1 is not an IMiDR.

11. The method of any one of the preceding claims, wherein the inhibitor of UHRF1 is a direct inhibitor of UHRF1.

12. The method of claim 11, wherein the direct inhibitor of UHRF1 is a direct inhibitor of a UHRF1 protein.

13. The method of claim 11 or claim 12, wherein the inhibitor of UHRF1 is a small molecule, aptamer, or antibody that binds to the UHRF1 protein.

14. The method of any one of claims 11-13, wherein the inhibitor of UHRF1 is idarubicin, mitoxantrone, berberine, daunorubicin, doxorubicin, mitoxantrone, pixantrone, 2,4-lutidine, or NSC232003.

15. The method of claim 11, wherein the inhibitor of UHRF1 is a direct inhibitor of a UHRF1 transcript.

16. The method of claim 15, wherein the inhibitor of UHRF1 comprises an oligonucleotide that hybridizes to the UHRF1 transcript.

17. The method of claim 15 or claim 16, wherein the inhibitor of UHRF1 comprises an siRNA, shRNA, or antisense oligonucleotide.

18. The method of any one of the preceding claims, wherein the multiple myeloma cells express a UHRF1 transcript or protein.

19. The method of claim 18, wherein the multiple myeloma expresses elevated levels of the UHRF1 transcript or protein relative to a reference level of UHRF1 transcript or protein.

20. The method of claim 18 or claim 19, wherein the UHRF1 transcript or protein expression is determined by a method comprising in situ hybridization, PCR, RNA sequencing, fluorescence in situ hybridization (FISH), mixed ligation probe assays, RT-qPCR, western blot, ELISA, or mass spectrometry.

21. A method of selecting a subject with multiple myeloma for treatment with a UHRF1 inhibitor, comprising determining the level of UHRF1 in a sample obtained from the subject, wherein if the level of UHRF1 in the sample is above a reference level, the subject is selected for treatment with a UHRF1 inhibitor.

22. A method of predicting whether a subject with multiple myeloma will benefit from treatment with a UHRF1 inhibitor, comprising determining whether the subject has a level of UHRF1 above a reference level.

23. The method of claim 22, wherein the level of UHRF1 is determined in a sample from the subject.

24. The method of any one of claims 21-23, wherein the level of UHRF1 is the level of a UHRF1 transcript or protein.

25. The method of claim 24, wherein the UHRF1 transcript or protein level is determined by a method comprising in situ hybridization, PCR, RNA sequencing, fluorescence in situ hybridization (FISH), mixed ligation probe assays, RT-qPCR, western blot, ELISA, or mass spectrometry.

26. The method of any one of claims 21-25, wherein the method further comprises administering to the subject a therapeutically effective amount of an inhibitor of UHRF1.

27. The method of claim 26, wherein the multiple myeloma is resistant to at least one IMiD®.

28. The method of claim 27, wherein the multiple myeloma is resistant to at least one IMiD® selected from lenalidomide, thalidomide, pomalidomide, avadomide, and iberdomide.

29. A method of treating multiple myeloma, comprising administering to a subject with multiple myeloma a therapeutically effective amount of an inhibitor of Ubiquitin-Like with PHD and Ring Finger Domains 1 (UHRF1) and at least one additional therapeutic agent.

30. The method of claim 29, wherein the multiple myeloma is resistant to at least one IMiD®.

31. The method of claim 30, wherein the multiple myeloma is resistant to at least one IMiD® selected from lenalidomide, thalidomide, pomalidomide, avadomide, and iberdomide.

32. The method of any one of claims 29-31, wherein at least one additional therapeutic agent is selected from a chemotherapy agent, a steroid, an immunomodulating agent, a proteasome inhibitor, a histone deacetylase inhibitor, an anti-CD38 antibody, an anti-SLAMF7 antibody, an antibody-drug conjugate, a nuclear export inhibitor, a BCMA-directed T-cell engager, an NK cell engager, and a CAR-T therapy.

33. The method of any one of claims 29-32, wherein at least one additional therapeutic agent is selected from lenalidomide, thalidomide, pomalidomide, iberdomide, bortezomib, carfilzomib, ixazomib, panobinostat, melphalan, vincristine, cyclophosphamide, etoposide, doxorubicin, and bendamustine, dexamethasone, prednisone, daratumumab, isatuximab, elotuzumab, belantamab mafodotin-blmf, Selinexor, pamidronate, zoledronic acid, and denosumab.

34. The method of any one of claims 29-33, wherein the at least one additional therapeutic agent is selected from:
a) lenalidomide;
b) iberdomide;
c) (S)-4-(4-(4-(((2-(2,6-dioxopiperidin-3-yl)-1-oxoisoindolin-4-yl)oxy)methyl)benzyl) piperazin-1-yl)-3-fluorobenzonitrile;
d) (i) lenalidomide, pomalidomide, or thalidomide; and (ii) dexamethasone;
e) (i) carfilzomib, ixazomib, or bortezomib; (ii) lenalidomide; and (iii) dexamethasone;
f) (i) bortezomib or carfilzomib; (ii) cyclophosphamide; and (iii) dexamethasone;
g) (i) elotuzumab or daratumumab; (ii) lenalidomide; and (iii) dexamethasone;
h) bortezomib, liposomal doxorubicin, and dexamethasone;
i) panobinostat, bortezomib, and dexamethasone;
j) elotuzumab, bortezomib, and dexamethasone;
k) melphalan and prednisone, with or without thalidomide or bortezomib;
l) vincristine, doxorubicin, and dexamethasone;
m) dexamethasone, cyclophosphamide, etoposide, and cisplatin; and
n) dexamethasone, thalidomide, cisplatin, doxorubicin, cyclophosphamide, and etoposide, with or without bortezomib.

35. The method of any one of claims 26-34, wherein the inhibitor of UHRF1 is not an IMiD®.

36. The method of any one of claims 26-35, wherein the inhibitor of UHRF1 is a direct inhibitor of UHRF1.

37. The method of claim 36, wherein the direct inhibitor of UHRF1 is a direct inhibitor of a UHRF1 protein.

38. The method of claim 36 or claim 37, wherein the inhibitor of UHRF1 is a small molecule, aptamer, or antibody that binds to the UHRF1 protein.

39. The method of any one of claims 36-38, wherein the inhibitor of UHRF1 is idarubicin, mitoxantrone, berberine, daunorubicin, doxorubicin, mitoxantrone, pixantrone, 2,4-lutidine, or NSC232003.

40. The method of claim 36, wherein the inhibitor of UHRF1 is a direct inhibitor of a UHRF1 transcript.

41. The method of claim 40, wherein the inhibitor of UHRF1 comprises an oligonucleotide that hybridizes to the UHRF1 transcript.

42. The method of claim 40 or claim 41, wherein the inhibitor of UHRF1 comprises an siRNA, shRNA, or antisense oligonucleotide.

43. The method of any one of claims 21-42, wherein the sample is a blood sample, plasma sample, or multiple myeloma sample.

44. The method of claim 43, wherein the sample is a multiple myeloma sample.
